(19) United States
(12) Patent Application Publication
(10) Pub. No.: US 2026/0315462 A1
(43) Pub. Date: Oct. 15, 2026

(54) METHODS OF TREATING RELAPSING MULTIPLE SCLEROSIS USING AN INHIBITOR OF BRUTON'S TYROSINE KINASE

(71) Applicant: Genentech, Inc., South San Francisco, CA (US)

(72) Inventors: Alexandra Lee Goodyear, South San Francisco, CA (US); John Nolan Ratchford, South San Francisco, CA (US); Julie Anne Napieralski, South San Francisco, CA (US); David Brian Clayton, South San Francisco, CA (US); Xiaoye MA, South San Francisco, CA (US); Qi QI, South San Francisco, CA (US); Denison John Kuruvilla, South San Francisco, CA (US); Christopher Todd Harp, South San Francisco, CA (US)

(21) Appl. No.: 19/484,803

(22) PCT Filed: May 14, 2024
(86) PCT No.: PCT/US2024/029225

§ 371 Date: Nov. 14, 2025

Related U.S. Application Data
(60) Provisional application No. 63/502,460, filed on May 16, 2023.
(60) Provisional application No. 63/505,747, filed on Jun. 2, 2023.
(60) Provisional application No. 63/588,389, filed on Oct. 6, 2023.

(51) Int. Cl. A61K 31/4985; A61P 25/28

(52) U.S. Cl. CPC A61K 31/4985; A61P 25/28

(57) ABSTRACT

Provided herein are methods of treating Relapsing Multiple Sclerosis (RMS) with fenebrutinib, an inhibitor of Bruton's tyrosine kinase (BTK).

### CROSS-REFERENCE TO RELATED APPLICATIONS

This application claims the benefit of priority of U.S. Provisional Application Ser. No. 63/502,460 filed May 16, 2023; U.S. Provisional Application Ser. No. 63/505,747 filed Jun. 2, 2023; and U.S. Provisional Application Ser. No. 63/588,389 filed Oct. 6, 2023, the disclosures of each of which are incorporated herein by reference in their entireties.

### FIELD

The present disclosure relates to methods of treating relapsing multiple sclerosis (RMS) using an inhibitor of Bruton's tyrosine kinase (BTK).

### BACKGROUND

Bruton's tyrosine kinase (BTK) is essential for the differentiation and activity of B cells during immune system ontogeny and normal adaptive immune responses. BTK is activated by phosphatidylinositol 3-kinase-dependent plasma membrane recruitment and phosphorylation on tyrosine Y551 by the Src-family kinase Lyn. Autophosphorylation and activation also occurs on tyrosine Y223 in a BTK-specific manner. Once activated, BTK induces PLCγ2- and Ca²⁺-dependent signaling, which leads to the activation of NF-κB- and NFAT-dependent pathways; this in turn leads to cellular activation and differentiation (Niiro H, Clark E A., Nat Rev Immunol 2002, 2:945-56). BTK is a key mediator of B-cell receptor (BCR) signaling in B-cells and Fcγ receptor signaling in myeloid cells. Fenebrutinib, as a BTK inhibitor, effectively blocks B-cell activation and proliferation as well as myeloid effector functions.

Multiple Sclerosis: Multiple sclerosis (MS) is a chronic, inflammatory, demyelinating, and degenerative disease of the CNS that affects approximately 900,000 people in the United States and 2.8 million worldwide (Wallin et al. Neurology 2019; 92: e1029-40; Walton et al. Mult Scler J 2020; 26:1816-21). It is primarily a disease of young adults, with 70%-80% of patients having an age of onset (i.e., initial clinical presentation to a physician) between 20 and 40 years, and has a gender bias influenced by the phenotype, with approximately up to 64%-70% of diagnosed patients being women (Anderson et al. Ann Neurol 1992, 31:333-6; Noonan et al. Neurology 2002, 58:136-8).

Traditionally, MS is classified into three clinical phenotypes, one of which is relapsing MS (RMS). Without wishing to be bound by any theory, disability progression across the spectrum of MS might occur as a result of two concurrent inflammatory mechanisms: active inflammation and chronic compartmentalized inflammation. These two types of inflammation may contribute in different extents across the different types and stages of MS.

RMS is associated with an active inflammatory mechanism characterized by focal, bulk T-cell, and B-cell invasion and blood brain barrier leakage that give rise to classic active demyelinating plaques in the white matter. Chronic compartmentalized inflammation is thought to be responsible for an increase in disability that occurs independently of relapses or disease activity and is characterized by demyelination and axonal loss (progression biology; Lassmann et al. 2019). While this aspect of inflammation is considered the hallmark of progressive forms of MS, RMS phenotypes also harbor signs of progression biology/chronic compartmentalized inflammation, which expresses itself as a chronic and slow accumulation of T cells and B cells without leakage of the blood brain barrier and may create subpial-demyelinated lesions in the cerebral and cerebellar cortex, as well as a slow expansion of pre-existing lesions in the white matter and diffuse chronic inflammation in the normal appearing white and gray matter (Lassmann 2018). Finally, the role of the myeloid lineage cells, including macrophages and microglia, may also impact both pathological and clinical outcomes (Absinta et al, 2020).

In vitro cell-based experiments suggest that antagonism of BTK with fenebrutinib leads to inhibition of BCR-dependent B-cell proliferation and a reduction of inflammatory cytokine production from myeloid cells (including tumor necrosis factor-α [TNF-α]). Myeloid effector functions are triggered by immune complexes in vitro, and increasing evidence suggests that B cells and myeloid/microglia may be central to the immunopathology of MS (Hauser et al.; N Engl J Med 2017; 376:221-34; Montalban et al. N Engl J Med. 2019; 380:2406-17; Howell et al. 2010 Journal of neuropathology and experimental neurology 2010; 69:1017-33). BTK inhibition has direct effects on myeloid lineage cells. As a result, there is a potential for BTK inhibition to affect microglia that are associated with the pathological hallmark of MS disease progression independent of relapse.

BTK inhibitors (BTKi) represent a new class of molecule being investigated for treatment of MS, and to date no BTKi has been approved by the FDA or EMA for treating any form of MS. While multiple BTKi are being studied in clinical trials to treat various forms of MS, this group of molecules is structurally and pharmacologically diverse, including differences in selectivity, reversibility, covalent vs. noncovalent action, and CNS penetrance (Schneider, R., Oh, J. Curr Neurol Neurosci Rep 22, 721-734, 2022). The different drug metabolism and pharmacokinetic (DMPK) properties and dosing schedules add another layer of complexity. These various factors (alone or in combination) render the molecules not interchangeable, as they may lead to differences in safety, efficacy, or both, in treating MS

There is still a need for effective treatments of RMS. Provided herein are methods of treating RMS, including methods of reducing the number of new T1 Gd+ lesions, and new or enlarging T2-weighted lesions, using fenebrutinib or a pharmaceutically acceptable salt thereof.

### SUMMARY OF THE DISCLOSURE

Provided herein are methods and uses of a BTK inhibitor, fenebrutinib, or a pharmaceutically acceptable salt of fenebrutinib, for treating Relapsing Multiple Sclerosis (RMS).

Provided herein are methods of treating relapsing multiple sclerosis (RMS) in a subject in need thereof, comprising administering to the subject about 200 mg fenebrutinib twice daily, or an equivalent amount of a pharmaceutically acceptable salt thereof, and wherein the treatment comprises any one of:
reducing the total number of new gadolinium enhancing T1 lesions in the subject over 12 weeks after beginning administration, as evaluated at 4, 8, and 12 weeks;
reducing the total number of new gadolinium enhancing T1 lesions at 4 weeks after beginning administration;
reducing the total number of new gadolinium enhancing T1 lesions at 8 weeks after beginning administration;
reducing the total number of new gadolinium enhancing T1 lesions as evaluated at weeks 8 and 12 after beginning administration;
reducing by at least 60% the total number of new gadolinium enhancing T1 lesions at 12 weeks after beginning administration;
reducing the rate of developing new gadolinium enhancing T1 lesions;
preventing the development of new gadolinium enhancing T1 lesions;
reducing the total number of new or enlarging T2-weighted lesions over 12 weeks after beginning administration, as evaluated at 4, 8, and 12 weeks;
reducing the total number of new or enlarging T2-weighted lesions at 4 weeks after beginning administration;
reducing the total number of new or enlarging T2-weighted lesions at 8 weeks after beginning administration;
reducing the total number of new or enlarging T2-weighted lesions as evaluated at weeks 8 and 12 after beginning administration;
reducing by at least 60% the total number of new or enlarging T2-weighted lesions at 12 weeks after beginning administration;
reducing the rate of developing new or enlarging T2-weighted lesions;
preventing the development of new or enlarging T2-weighted lesions; or
increasing the odds that a subject will be free from both (a) any new gadolinium enhancing T1 lesions; and (b) any new or enlarging T2-weighted lesions;
or any combinations thereof.

In some aspects of the above methods, the reduction, prevention, or increasing is relative to a subject with RMS who is not administered fenebrutinib, or a pharmaceutically acceptable salt thereof. In certain aspects, this subject not administered fenebrutinib, or a pharmaceutically acceptable salt thereof, is the same subject, but prior to beginning administration of fenebrutinib or a pharmaceutically acceptable salt thereof; such as the same subject evaluated in the 12 weeks, 6 months, or 12 months immediately preceding beginning administration. In some aspects, the subject not administered fenebrutinib, or a pharmaceutically acceptable salt thereof, is a different subject with RMS who is not administered fenebrutinib or a pharmaceutically acceptable salt thereof; in certain aspects, the subject is not administered any RMS therapy.

Further provided herein is fenebrutinib or a pharmaceutically acceptable salt thereof for use in treating RMS in a subject in need thereof, wherein treating comprises administering to the subject about 200 mg fenebrutinib twice daily, or an equivalent amount of a pharmaceutically acceptable salt thereof, and wherein treating further comprises any one of:
reducing the total number of new gadolinium enhancing T1 lesions in the subject over 12 weeks after beginning administration, as evaluated at 4, 8, and 12 weeks;
reducing the total number of new gadolinium enhancing T1 lesions at 4 weeks after beginning administration;
reducing the total number of new gadolinium enhancing T1 lesions at 8 weeks after beginning administration;
reducing the total number of new gadolinium enhancing T1 lesions as evaluated at weeks 8 and 12 after beginning administration;
reducing by at least 60% the total number of new gadolinium enhancing T1 lesions at 12 weeks after beginning administration;
reducing the rate of developing new gadolinium enhancing T1 lesions;
preventing the development of new gadolinium enhancing T1 lesions;
reducing the total number of new or enlarging T2-weighted lesions over 12 weeks after beginning administration, as evaluated at 4, 8, and 12 weeks;
reducing the total number of new or enlarging T2-weighted lesions at 4 weeks after beginning administration;
reducing the total number of new or enlarging T2-weighted lesions at 8 weeks after beginning administration;
reducing the total number of new or enlarging T2-weighted lesions as evaluated at weeks 8 and 12 after beginning administration;
reducing by at least 60% the total number of new or enlarging T2-weighted lesions at 12 weeks after beginning administration;
reducing the rate of developing new or enlarging T2-weighted lesions;
preventing the development of new or enlarging T2-weighted lesions; or
increasing the odds that a subject will be free from both (a) any new gadolinium enhancing T1 lesions; and (b) any new or enlarging T2-weighted lesions;
or any combinations thereof.

In some aspects of the above compound for use, the reduction, prevention, or increasing is relative to a subject with RMS who is not administered fenebrutinib, or a pharmaceutically acceptable salt thereof. In certain aspects, this subject not administered fenebrutinib, or a pharmaceutically acceptable salt thereof, is the same subject, but prior to beginning administration of fenebrutinib or a pharmaceutically acceptable salt thereof; such as the same subject evaluated in the 12 weeks, 6 months, or 12 months immediately preceding beginning administration. In some aspects, the subject not administered fenebrutinib, or a pharmaceutically acceptable salt thereof, is a different subject with RMS who is not administered fenebrutinib or a pharmaceutically acceptable salt thereof; in certain aspects, this different subject is not administered any RMS therapy.

Also provided is a compound for use in the manufacture of a medicament for treatment of relapsing multiple sclerosis (RMS) in a subject in need thereof, wherein the compound is fenebrutinib or a pharmaceutically acceptable salt thereof, and the treatment comprises any of the methods as provided herein.

### DESCRIPTION OF THE DRAWINGS

FIG. 1 is a diagram of the clinical trial design, for the clinical trial described by Example 1.

FIG. 2 provides bar graphs summarizing adjusted rate (combined Weeks 4, 8 and 12) and adjusted mean number (separate time points) of new T1 Gd+ lesions observed in the patients administered fenebrutinib, compared to those administered placebo. For the separate time points, the number of patients refers to the patients with evaluable MRI scans at that visit.

FIG. 3 provides bar graphs summarizing adjusted rate (combined Weeks 4, 8 and 12) and adjusted mean number (separate time points) of new/enlarging T2-weighted lesions observed in the patients administered fenebrutinib, compared to those administered placebo. For the separate time points, the number of patients refers to the patients with evaluable MRI scans at that visit.

FIG. 4 provides bar graphs summarizing adjusted rate (combined Weeks 4, 8 and 12) and adjusted mean number (separate time points) of new T1 hypointense lesions observed in the patients administered fenebrutinib, compared to those administered placebo. For the separate time points, the number of patients refers to the patients with evaluable MRI scans at that visit.

FIG. 5 is a plot of fenebrutinib concentration in cerebrospinal fluid (CSF) samples taken from 11 patients after 12 weeks of continuous fenebrutinib administration. The results are plotted against the IC50 of fenebrutinib (active inhibitory threshold) and the IC90 of fenebrutinib (maximal inhibitory threshold) as evaluated in different in vitro assays.

### DETAILED DESCRIPTION

Provided herein are methods and uses fenebrutinib, or a pharmaceutically acceptable salt of fenebrutinib, for treating Relapsing Multiple Sclerosis (RMS).

Fenebrutinib is a compound of the formula:

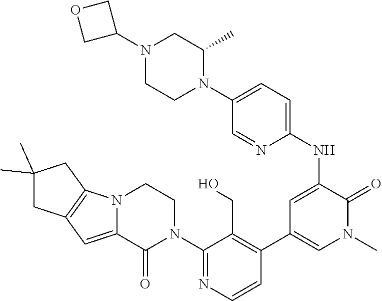 [SMILES: C[C@H]1CN(C2COC2)CCN1c1ccc(Nc2cc(-c3ccnc(N4CCn5c(cc6c5CC(C)(C)C6)C4=O)c3CO)cn(C)c2=O)nc1]

and is also known by the following names:
GDC-0853;
(6²S)-2³-(hydroxymethyl)-1⁷,1⁷,3¹,6²-tetramethyl-1³,1⁴,1⁷,1⁸-tetrahydro-4-aza-1 (2)-cyclopenta[4,5]pyrrolo[1,2-a]pyrazina-6 (1,4)-piperazina-2 (2,4),3 (3,5),5 (2,5)-tripyridina-7 (3)-oxetanaheptaphane-11 (16H),36 (31H)-dione; and
(S)-2-(3′-(hydroxymethyl)-1-methyl-5-((5-(2-methyl-4-(oxetan-3-yl) piperazin-1-yl)pyridin-2-yl)amino)-6-oxo-1,6-dihydro-[3,4′-bipyridin]-2′-yl)-7,7-dimethyl-2,3,4,6,7,8-hexahydro-1H-cyclopenta[4,5]pyrrolo[1,2-a]pyrazin-1-one.

The R enantiomer of the compound is: (R)-2-(3′-(hydroxymethyl)-1-methyl-5-((5-(2-methyl-4-(oxetan-3-yl)piperazin-1-yl)pyridin-2-yl)amino)-6-oxo-1,6-dihydro-[3,4′-bipyridin]-2′-yl)-7,7-dimethyl-2,3,4,6,7,8-hexahydro-1H-cyclopenta[4,5]pyrrolo[1,2-a]pyrazin-1-one.

Fenebrutinib is a highly selective, orally administered, reversible inhibitor of BTK. U.S. Pat. No. 8,716,274, which is hereby incorporated by reference in its entirety, discloses classes of heteroaryl pyridine and aza-pyridone compounds useful for inhibiting Btk, including fenebrutinib. WO 2017/148837, which is hereby incorporated by reference in its entirety, discloses solid forms and formulations of fenebrutinib and pharmaceutically acceptable salts thereof.

### I. Definitions

It is to be understood that the terminology used herein is for the purpose of describing particular embodiments only, and is not intended to be limiting.

As used in this specification, including the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se. In some embodiments, the term “about” refers to a range of plus or minus 10% for the respective value. In some embodiments, the term “about” refers to a range of plus or minus 5% for the respective value. In some embodiments, the term “about” refers to a range of plus or minus 2% for the respective value. In some embodiments, the term “about” refers to a range of plus or minus 1% for the respective value.

It is understood that aspects and embodiments of the disclosure described herein include “comprising,” “consisting,” and “consisting essentially of” aspects and embodiments.

The “Expanded Disability Status Scale” (EDSS) is a measure for quantifying changes in the disability level of a subject with MS over time. The EDSS is based on a standardized neurological examination, incorporating functional systems (visual, brainstem, pyramidal, cerebellar, sensory, bowel and bladder, and cerebral [or mental]) that are rated and then scored as a functional systems score (FSS), and ambulation, which is scored as ambulation score. Each FSS is an ordinal clinical rating scale ranging from 0 to 5 or 6 and an ambulation score that is rated from 0 to 16. These ratings are then used in conjunction with observations, as well as information, concerning ambulation and use of assistive devices to determine the total EDSS score. The EDSS is a disability scale that ranges in 0.5-point steps from 0 (normal) to 10.0 (death) (Kurtzke 1983; Kappos 2011). In some embodiments of the methods provided herein, sexual dysfunction and fatigue are not included in the EDSS score.

The term “lesions” as used herein refers to lesions observed on an MRI of the central nervous system of a subject. In the Examples described herein, the imaging is of the brain. These lesions can include gadolinium-enhancing T1 lesions; and new or enlarging T2-weighted lesions. These types of lesions are observed using different MRI techniques. Gadolinium enhancing T1-imaging may be used to image inflammation in acute MS lesions. Inflammation can lead to higher permeability in the blood-brain barrier, increasing the ability for a gadolinium contrast agent to cross the barrier and increasing contrast enhancement in gadolinium-enhanced T1 imaging. Gadolinium-enhanced T1 lesions may be the earliest change detectible in the development of most new lesions, indicate active inflammation, and may resolve over time as inflammation subsides. T2-weighted imaging is used to evaluate the total lesion load or lesion burden on a subject, including when the subject is not having acute inflammation. In an acute phase of MS, T2 lesions are often associated with T1 Gd+ lesions and may shrink in size over time as edema resolves. T2 lesions may change in signal intensity and size over time, and depending on the presence of active inflammation, but also can be a permanent mark of a prior inflammatory event. Thus, the number of new and enlarging T2-weighted lesions in a subject may be one method of monitoring progression over a period of time, including throughout the disease; and may be use to evaluate treatment response. New T2 lesions and Gd+ T1 lesions may be observable before apparent clinical signs and symptoms. The terms “T1 Gd+ lesions,” “Gd+ T1 lesions”, “gadolinium-enhancing T1 lesions”, and “Gd+ enhancing T1-weighted lesions” may be used interchangeably herein, and other permutations of said phrasing may also be possible. Monitoring of MS may also include evaluation of T1 hypointense lesions. T1 hypointense lesions may appear as dark or black on the MRI image, and are sometimes called “black holes”. T1 hypointense lesions may appear during the acute phase as a result of edema and demyelination, and these lesions may disappear as inflammation resolves. Persistent T1 hypointense lesions observed during the chronic phase represent irreversible axonal loss and correlate with disease progression and clinical disability.

The term “pharmaceutical formulation” refers to a preparation which is in such form as to permit the biological activity of the active ingredient to be effective, and which contains no additional components which are unacceptably toxic to a subject to which the formulation would be administered. In some embodiments, such formulations are sterile. “Pharmaceutically acceptable” excipients (vehicles, additives) are those which can reasonably be administered to a subject mammal to provide an effective dose of the active ingredient employed.

As used herein, the term “treatment” refers to clinical intervention designed to alter the natural course of the individual or cell being treated during the course of clinical pathology. Desirable effects of treatment may include decreasing the rate of disease progression, ameliorating or palliating the disease state, and remission or improved prognosis. In some embodiments, two or more of such effects are achieved. In some embodiments, an individual is successfully “treated” if one or more symptoms associated with their disease or disorder is diminished; the disease or disorder is made more tolerable to the subject; the rate of degeneration or decline, or rate of disease or disorder development is slowed or stopped; the progression of the disease or disorder is slowed or stopped; or the final point of degeneration is less debilitating. For example, an individual is successfully “treated” if one or more symptoms associated with the disease (e.g., MS) are mitigated or eliminated, including, but not limited to, decreasing symptoms resulting from the disease, increasing the quality of life of those suffering from the disease, decreasing the dose of other medications required to treat the disease, and/or prolonging survival of individuals. Treatment of certain diseases or disorders may in some embodiments include, but is not limited to, specific clinical or other endpoints such as those described in the Examples provided herein.

Reduction of “by at least” may also be rephrased as “greater than”. For example, a reduction of new gadolinium enhancing T1 lesions “by at least 80%” may also be phrased as a reduction of new gadolinium enhancing T1 lesions wherein the reduction “is greater than 80%”.

Some embodiments described herein refer to providing a dose of fenebrutinib, or an equivalent amount of a pharmaceutically acceptable salt thereof. It would be clear to one of skill in the art how to calculate a corresponding amount of a pharmaceutical salt form of fenebrutinib, taking into account the difference in molecular weight between the free form of fenebrutinib and a salt form. For example, in some embodiments provided herein, a subject is administered about 400 mg daily of fenebrutinib (as two, 200 mg doses), or a pharmaceutically acceptable salt thereof. If a pharmaceutically acceptable salt form is administered in such embodiments, due to the salt form having a higher molecular weight than the free form of fenebrutinib, the total weight of the pharmaceutically acceptable salt of fenebrutinib administered daily is greater than 400 mg, but corresponds to about 400 mg of the free form of fenebrutinib.

A “subject” for purposes of treatment refers to any animal classified as a mammal, including humans, domestic and farm animals, and zoo, sports, or pet animals, such as dogs, horses, cats, cows, etc. In some embodiments of the methods provided herein, the subject is human. In some embodiments, the subject is a patient.

“Prior to beginning administration” may include, for example, on the same day as, but before the actual administration of, the first dose of fenebrutinib or pharmaceutically acceptable salt thereof is administered; or within one week prior to the first dose; or within two weeks prior to the first dose; or within three weeks prior to the first dose; or within four weeks prior to the first dose; or within five weeks prior to the first dose; or within six weeks prior to the first dose; or within greater than six weeks prior to the first dose; or between 1 and 28 days prior to the first dose; or within 0 to 28 days prior to the first dose. In certain embodiments, this period of time may also be referred to as “baseline”. Thus, in some embodiments, baseline may include within one week prior to administering the first dose of fenebrutinib or a pharmaceutically acceptable salt thereof, including the same day just prior to administration. In other embodiments, baseline includes within one month, or within 0 to 28 days, or within six weeks prior to the first dose of fenebrutinib or a pharmaceutically acceptable salt thereof. In some embodiments, baseline is within 12 weeks, or within 6 months, or within 12 months prior to the first dose of fenebrutinib or a pharmaceutically acceptable salt thereof.

### II. Methods of Treatment

Provided herein are methods of treating a subject with relapsing multiple sclerosis (RMS), comprising administering to the subject about 200 mg fenebrutinib twice daily, or an equivalent amount of a pharmaceutically acceptable salt thereof. In some embodiments, provided herein is fenebrutinib or a pharmaceutically acceptable salt thereof for use in the treatment of RMS in a subject in need thereof, comprising administering to the subject about 200 mg of fenebrutinib twice daily, or an equivalent amount of a pharmaceutically acceptable salt thereof. In still further embodiments, provided herein is fenebrutinib or a pharmaceutically acceptable salt thereof for use in manufacturing a medicament for the treatment of RMS in a subject in need thereof, wherein the subject is administered about 200 mg of fenebrutinib twice daily, or an equivalent amount of a pharmaceutically acceptable salt thereof. In certain embodiments provided herein, including methods, fenebrutinib for use, or fenebrutinib for use in the manufacture of a medicament, the effect on the subject (e.g., treatment of RMS) is evaluated through one or more clinical outcomes, such as by measuring gadolinium-enhancing T1 lesions (T1 Gd+ lesions), or new or enlarging T2-weighted lesions, or a disability score such as EDSS, or a combination thereof, in the subject. In some embodiments, treatment is illustrated through the reduction in number of lesions, reduction in the rate of lesions, or increasing the odds of not developing lesions, wherein the lesions may be gadolinium-enhancing T1 lesions, or new or enlarging T2-weighted lesions, or a combination thereof. In some embodiments, such reductions or decreases occur within 4 weeks of beginning administration, within 8 weeks of beginning administration, or within 12 weeks of beginning administration, or are evaluated at one or more time points therein (e.g, over 12 weeks as evaluated at 4, 8, and 12 weeks; or as observed at 8 and 12 weeks combined).

Provided herein is a method of treating relapsing multiple sclerosis (RMS) in a subject in need thereof, comprising administering to the subject about 200 mg fenebrutinib twice daily, or an equivalent amount of a pharmaceutically acceptable salt thereof, and wherein the treatment comprises any one of:
reducing the total number of new gadolinium enhancing T1 lesions in the subject over 12 weeks after beginning administration, as evaluated at 4, 8, and 12 weeks;
reducing the total number of new gadolinium enhancing T1 lesions at 4 weeks after beginning administration;
reducing the total number of new gadolinium enhancing T1 lesions at 8 weeks after beginning administration;
reducing the total number of new gadolinium enhancing T1 lesions as evaluated at weeks 8 and 12 after beginning administration;
reducing by at least 60% the total number of new gadolinium enhancing T1 lesions at 12 weeks after beginning administration;
reducing the rate of developing new gadolinium enhancing T1 lesions;
preventing the development of new gadolinium enhancing T1 lesions;
reducing the total number of new or enlarging T2-weighted lesions over 12 weeks after beginning administration, as evaluated at 4, 8, and 12 weeks;
reducing the total number of new or enlarging T2-weighted lesions at 4 weeks after beginning administration;
reducing the total number of new or enlarging T2-weighted lesions at 8 weeks after beginning administration;
reducing the total number of new or enlarging T2-weighted lesions as evaluated at weeks 8 and 12 after beginning administration;
reducing by at least 60% the total number of new or enlarging T2-weighted lesions at 12 weeks after beginning administration;
reducing the rate of developing new or enlarging T2-weighted lesions;
preventing the development of new or enlarging T2-weighted lesions; or
increasing the odds that a subject will be free from both (a) any new gadolinium enhancing T1 lesions; and (b) any new or enlarging T2-weighted lesions;
or any combinations thereof.

Further provided herein is a method of treating relapsing multiple sclerosis (RMS) in a subject in need thereof, comprising administering to the subject about 200 mg fenebrutinib twice daily, or an equivalent amount of a pharmaceutically acceptable salt thereof, and wherein the treatment comprises any one of:
reducing the total number of new T1 hypointense lesions in the subject at 12 weeks after beginning administration;
reducing the total number of new T1 hypointense lesions at 8 weeks after beginning administration.
reducing the total number of new T1 hypointense lesions at 4 weeks after beginning administration;
reducing the total number of new T1 hypointense lesions at 12 weeks after beginning administration, as evaluated at 4, 8, and 12 weeks;
reducing the rate of developing new T1 hypointense lesions;
reducing the rate of developing new T1 hypointense lesions, wherein the rate is evaluated over a period of 4 weeks, 8 weeks, or 12 weeks, after beginning administration;
preventing the development of new T1 hypointense lesions; or
preventing the development of new T1 hypointense lesions over a period of 4 weeks, 8 weeks, or 12 weeks, after beginning administration;
or any combinations thereof.

In some aspects of any of the above methods, or any method disclosed herein, the reduction, prevention, or increasing is relative to a subject with RMS who is not administered fenebrutinib, or a pharmaceutically acceptable salt thereof. In certain aspects, this subject not administered fenebrutinib, or a pharmaceutically acceptable salt thereof, is the same subject, but prior to beginning administration of fenebrutinib or a pharmaceutically acceptable salt thereof; such as the same subject evaluated in the 12 weeks, 6 months, or 12 months immediately preceding beginning administration. In some aspects, the subject not administered fenebrutinib, or a pharmaceutically acceptable salt thereof, is a different subject with RMS who is not administered fenebrutinib or a pharmaceutically acceptable salt thereof; in certain aspects, this different subject is not administered any RMS therapy.

Further provided herein is fenebrutinib or a pharmaceutically acceptable salt thereof for use in treating RMS in a subject in need thereof, wherein treating comprises administering to the subject about 200 mg fenebrutinib twice daily, or an equivalent amount of a pharmaceutically acceptable salt thereof, and wherein treating further comprises any one of:
reducing the total number of new gadolinium enhancing T1 lesions in the subject over 12 weeks after beginning administration, as evaluated at 4, 8, and 12 weeks;
reducing the total number of new gadolinium enhancing T1 lesions at 4 weeks after beginning administration;
reducing the total number of new gadolinium enhancing T1 lesions at 8 weeks after beginning administration;
reducing the total number of new gadolinium enhancing T1 lesions as evaluated at weeks 8 and 12 after beginning administration;
reducing by at least 60% the total number of new gadolinium enhancing T1 lesions at 12 weeks after beginning administration;
reducing the rate of developing new gadolinium enhancing T1 lesions;
preventing the development of new gadolinium enhancing T1 lesions;
reducing the total number of new or enlarging T2-weighted lesions over 12 weeks after beginning administration, as evaluated at 4, 8, and 12 weeks;
reducing the total number of new or enlarging T2-weighted lesions at 4 weeks after beginning administration;
reducing the total number of new or enlarging T2-weighted lesions at 8 weeks after beginning administration;
reducing the total number of new or enlarging T2-weighted lesions as evaluated at weeks 8 and 12 after beginning administration;
reducing by at least 60% the total number of new or enlarging T2-weighted lesions at 12 weeks after beginning administration;
reducing the rate of developing new or enlarging T2-weighted lesions;
preventing the development of new or enlarging T2-weighted lesions; or
increasing the odds that a subject will be free from both (a) any new gadolinium enhancing T1 lesions; and (b) any new or enlarging T2-weighted lesions;
or any combinations thereof.

Further provided herein is fenebrutinib or a pharmaceutically acceptable salt thereof for use in treating RMS in a subject in need thereof, wherein treating comprises administering to the subject about 200 mg fenebrutinib twice daily, or an equivalent amount of a pharmaceutically acceptable salt thereof, and wherein treating further comprises any one of:
reducing the total number of new T1 hypointense lesions in the subject at 12 weeks after beginning administration;
reducing the total number of new T1 hypointense lesions at 8 weeks after beginning administration.
reducing the total number of new T1 hypointense lesions at 4 weeks after beginning administration;
reducing the total number of new T1 hypointense lesions at 12 weeks after beginning administration, as evaluated at 4, 8, and 12 weeks;
reducing the rate of developing new T1 hypointense lesions;
reducing the rate of developing new T1 hypointense lesions, wherein the rate is evaluated over a period of 4 weeks, 8 weeks, or 12 weeks, after beginning administration;
preventing the development of new T1 hypointense lesions; or
preventing the development of new T1 hypointense lesions over a period of 4 weeks, 8 weeks, or 12 weeks, after beginning administration;
or any combinations thereof.

In some aspects of any of the above compounds for use, or any compound for use disclosed herein, the reduction, prevention, or increasing is relative to a subject with RMS who is not administered fenebrutinib, or a pharmaceutically acceptable salt thereof. In certain aspects, this subject not administered fenebrutinib, or a pharmaceutically acceptable salt thereof, is the same subject, but prior to beginning administration of fenebrutinib or a pharmaceutically acceptable salt thereof; such as the same subject evaluated in the 12 weeks, 6 months, or 12 months immediately preceding beginning administration. In some aspects, the subject not administered fenebrutinib, or a pharmaceutically acceptable salt thereof, is a different subject with RMS who is not administered fenebrutinib or a pharmaceutically acceptable salt thereof; in certain aspects, this different subject is not administered any RMS therapy.

Also provided herein is fenebrutinib or a pharmaceutically acceptable salt thereof for use in the manufacture of a medicament for treating relapsing multiple sclerosis (RMS) in a subject in need thereof, wherein the medicament comprises about 200 mg fenebrutinib or an equivalent amount of a pharmaceutically acceptable salt thereof, and wherein the medicament is administered to the subject twice daily, and wherein treatment comprises any one of:
reducing the total number of new gadolinium enhancing T1 lesions in the subject over 12 weeks after beginning administration, as evaluated at 4, 8, and 12 weeks;
reducing the total number of new gadolinium enhancing T1 lesions at 4 weeks after beginning administration;
reducing the total number of new gadolinium enhancing T1 lesions at 8 weeks after beginning administration;
reducing the total number of new gadolinium enhancing T1 lesions as evaluated at weeks 8 and 12 after beginning administration;
reducing by at least 60% the total number of new gadolinium enhancing T1 lesions at 12 weeks after beginning administration;
reducing the rate of developing new gadolinium enhancing T1 lesions;
preventing the development of new gadolinium enhancing T1 lesions;
reducing the total number of new or enlarging T2-weighted lesions over 12 weeks after beginning administration, as evaluated at 4, 8, and 12 weeks;
reducing the total number of new or enlarging T2-weighted lesions at 4 weeks after beginning administration;
reducing the total number of new or enlarging T2-weighted lesions at 8 weeks after beginning administration;
reducing the total number of new or enlarging T2-weighted lesions as evaluated at weeks 8 and 12 after beginning administration;
reducing by at least 60% the total number of new or enlarging T2-weighted lesions at 12 weeks after beginning administration;
reducing the rate of developing new or enlarging T2-weighted lesions;
preventing the development of new or enlarging T2-weighted lesions; or
increasing the odds that a subject will be free from both (a) any new gadolinium enhancing T1 lesions; and (b) any new or enlarging T2-weighted lesions;
or any combinations thereof.

Also provided herein is fenebrutinib or a pharmaceutically acceptable salt thereof for use in the manufacture of a medicament for treating relapsing multiple sclerosis (RMS) in a subject in need thereof, wherein the medicament comprises about 200 mg fenebrutinib or an equivalent amount of a pharmaceutically acceptable salt thereof, and wherein the medicament is administered to the subject twice daily, and wherein treatment comprises any one of:
reducing the total number of new T1 hypointense lesions in the subject at 12 weeks after beginning administration;
reducing the total number of new T1 hypointense lesions at 8 weeks after beginning administration.
reducing the total number of new T1 hypointense lesions at 4 weeks after beginning administration;
reducing the total number of new T1 hypointense lesions at 12 weeks after beginning administration, as evaluated at 4, 8, and 12 weeks;
reducing the rate of developing new T1 hypointense lesions;
reducing the rate of developing new T1 hypointense lesions, wherein the rate is evaluated over a period of 4 weeks, 8 weeks, or 12 weeks, after beginning administration;
preventing the development of new T1 hypointense lesions; or
preventing the development of new T1 hypointense lesions over a period of 4 weeks, 8 weeks, or 12 weeks, after beginning administration;
or any combinations thereof.

In some aspects of any of the above compound for use in the manufacture of a medicament, or any compound for use disclosed herein, the reduction, prevention, or increasing is relative to a subject with RMS who is not administered fenebrutinib, or a pharmaceutically acceptable salt thereof. In certain aspects, this subject not administered fenebrutinib, or a pharmaceutically acceptable salt thereof, is the same subject, but prior to beginning administration of fenebrutinib or a pharmaceutically acceptable salt thereof; such as the same subject evaluated in the 12 weeks, 6 months, or 12 months immediately preceding beginning administration. In some aspects, the subject not administered fenebrutinib, or a pharmaceutically acceptable salt thereof, is a different subject with RMS who is not administered fenebrutinib or a pharmaceutically acceptable salt thereof; in certain aspects, this different subject is not administered any RMS therapy.

Further provided herein is a method of treating Multiple Sclerosis (MS) in a subject in need thereof, comprising orally administering to the subject a sufficient amount of a BTK inhibitor to reach a cerebrospinal fluid (CSF) concentration greater than the active inhibitory threshold for inhibition of B-cell activation, inhibition of myeloid lineage cell activation, inhibition of BTK, or any combinations thereof, for said BTK inhibitor. Further provided herein is fenebrutinib or a pharmaceutically acceptable salt thereof for use in treating MS in a subject in need thereof, wherein treating comprises orally administering to the subject a sufficient amount of a BTK inhibitor to reach a cerebrospinal fluid (CSF) concentration greater than the active inhibitory threshold for inhibition of B-cell activation, inhibition of myeloid lineage cell activation, inhibition of BTK, or any combinations thereof, for said BTK inhibitor. Also provided herein is a small molecule BTK inhibitor for use in the manufacture of a medicament for treating MS in a subject in need thereof, wherein the medicament comprises sufficient BTK inhibitor such that daily oral administration to the subject reaches a cerebrospinal fluid (CSF) concentration greater than the active inhibitory threshold for inhibition of B-cell activation, inhibition of myeloid lineage cell activation, inhibition of BTK, or any combinations thereof, for said BTK inhibitor.

Each of the provided methods, uses, and treatments is described in greater detail herein.

### A. Gadolinium Enhancing T1 Lesions

In some embodiments, provided herein is a method of treating RMS in a subject in need thereof, comprising administering to the subject about 200 mg fenebrutinib twice daily, or an equivalent amount of a pharmaceutically acceptable salt thereof, wherein the treating comprises reducing the total number of new gadolinium enhancing T1 lesions over 12 weeks after beginning administration, as evaluated at 4, 8, and 12 weeks. In some embodiments, provided herein is a method of reducing the total number of new gadolinium enhancing T1 lesions in a subject with RMS in need thereof, comprising administering to the subject about 200 mg fenebrutinib twice daily, or an equivalent amount of a pharmaceutically acceptable salt thereof, and wherein the total number is reduced over 12 weeks after beginning administration, as evaluated at 4, 8, and 12 weeks. Further provided is fenebrutinib or a pharmaceutically acceptable salt thereof, for use in treating RMS in a subject in need thereof, comprising administering to the subject about 200 mg fenebrutinib twice daily, or an equivalent amount of a pharmaceutically acceptable salt thereof, wherein the treating comprises reducing the total number of new gadolinium enhancing T1 lesions over 12 weeks after beginning administration, as evaluated at 4, 8, and 12 weeks. Still further provided is fenebrutinib or a pharmaceutically acceptable salt thereof, for use in reducing the total number of new gadolinium enhancing T1 lesions in a subject with RMS in need thereof, comprising administering to the subject about 200 mg fenebrutinib twice daily, or an equivalent amount of a pharmaceutically acceptable salt thereof, and wherein the total number is reduced over 12 weeks after beginning administration, as evaluated at 4, 8, and 12 weeks. In some such embodiments of the methods and uses provided herein, the total number of new gadolinium enhancing T1 lesions over 12 weeks after beginning administration, as evaluated at 4, 8, and 12 weeks, is reduced by at least 30%, or by at least 40%, or by at least 50%. In some embodiments, the reduction is by at least 60%. In certain embodiments, the reduction is by at least 65%. In some embodiments, the reduction is by 69%. In certain embodiments, the reduction is a relative reduction as compared to if no fenebrutinib or pharmaceutically acceptable salt thereof were administered. In some embodiments, the subject is orally administered fenebrutinib as two tablets per day wherein each tablet comprises 200 mg fenebrutinib, or as four tablets per day wherein each tablet 100 mg fenebrutinib, for a total daily dose of 400 mg fenebrutinib. Further provided is the use of fenebrutinib or a pharmaceutically acceptable salt thereof, for the manufacture of a medicament for use in any of the methods or uses provided herein.

In other embodiments, the treatment comprises reducing the total number of new gadolinium enhancing T1 lesions within 4 weeks after beginning administration of fenebrutinib or an equivalent amount of a pharmaceutically acceptable salt thereof. Thus in some embodiments, provided herein is a method of treating RMS in a subject in need thereof, comprising administering to the subject about 200 mg fenebrutinib twice daily, or an equivalent amount of a pharmaceutically acceptable salt thereof, and wherein the treating comprises reducing the total number of new gadolinium enhancing T1 lesions at 4 weeks after beginning administration. In some such embodiments, provided herein is a method of reducing the total number of new gadolinium enhancing T1 lesions, comprising administering to the subject about 200 mg fenebrutinib twice daily, or an equivalent amount of a pharmaceutically acceptable salt thereof, wherein the reduction is at 4 weeks after beginning administration. Further provided is fenebrutinib or a pharmaceutically acceptable salt thereof, for use in treating RMS in a subject in need thereof, comprising administering to the subject about 200 mg fenebrutinib twice daily, or an equivalent amount of a pharmaceutically acceptable salt thereof, wherein the treating comprises reducing the total number of new gadolinium enhancing T1 lesions at 4 weeks after beginning administration. Still further provided is fenebrutinib or a pharmaceutically acceptable salt thereof, for use in reducing the total number of new gadolinium enhancing T1 lesions in a subject with RMS in need thereof, comprising administering to the subject about 200 mg fenebrutinib twice daily, or an equivalent amount of a pharmaceutically acceptable salt thereof, and wherein the total number is reduced at 4 weeks after beginning administration. In some such embodiments of the methods and uses provided herein, the total number of new gadolinium enhancing T1 lesions is reduced by at least 15% at 4 weeks. In some embodiments, reduction is by at least 20% at 4 weeks. In some embodiments, the reduction is by 22% at 4 weeks. In certain embodiments, the reduction is a relative reduction as compared to if no fenebrutinib or pharmaceutically acceptable salt thereof were administered. In some embodiments, fenebrutinib is orally administered. In some embodiments, the subject is orally administered fenebrutinib as two tablets per day wherein each tablet comprises 200 mg fenebrutinib, or as four tablets per day wherein each tablet 100 mg fenebrutinib, for a total daily dose of 400 mg fenebrutinib.

In still further embodiments, the treatment comprises reducing the total number of new gadolinium enhancing T1 lesions within 8 weeks after beginning administration. Thus in some embodiments, provided herein is a method of treating RMS in a subject in need thereof, comprising administering to the subject about 200 mg fenebrutinib twice daily, or an equivalent amount of a pharmaceutically acceptable salt thereof, wherein treating comprises reducing the total number of new gadolinium enhancing T1 lesions at 8 weeks after beginning administration. In some embodiments, provided herein is a method of reducing the total number of new gadolinium enhancing T1 lesions in a subject with RMS in need thereof, comprising administering to the subject about 200 mg fenebrutinib twice daily, or an equivalent amount of a pharmaceutically acceptable salt thereof, and wherein the total number is reduced at 8 weeks after beginning administration. Further provided is fenebrutinib or a pharmaceutically acceptable salt thereof, for use in treating RMS in a subject in need thereof, comprising administering to the subject about 200 mg fenebrutinib twice daily, or an equivalent amount of a pharmaceutically acceptable salt thereof, wherein the treating comprises reducing the total number of new gadolinium enhancing T1 lesions at 8 weeks after beginning administration. Still further provided is fenebrutinib or a pharmaceutically acceptable salt thereof, for use in reducing the total number of new gadolinium enhancing T1 lesions in a subject with RMS in need thereof, comprising administering to the subject about 200 mg fenebrutinib twice daily, or an equivalent amount of a pharmaceutically acceptable salt thereof, and wherein the total number is reduced at 8 weeks after beginning administration. In some such embodiments of the methods and uses provided herein, the total number of new gadolinium enhancing T1 lesions is reduced by at least 60% at 8 weeks. In some such embodiments, the reduction is at least 70% at 8 weeks. In some embodiments, the reduction is at least 80% at 8 weeks. In some embodiments, the reduction is at least 90% at 8 weeks. In some embodiments, the reduction is by 92% at 8 weeks. In certain embodiments, the reduction is a relative reduction as compared to if no fenebrutinib or pharmaceutically acceptable salt thereof were administered. In some embodiments, fenebrutinib is orally administered. In some embodiments, the subject is orally administered fenebrutinib as two tablets per day wherein each tablet comprises 200 mg fenebrutinib, or as four tablets per day wherein each tablet 100 mg fenebrutinib, for a total daily dose of 400 mg fenebrutinib.

In yet further embodiments, the treatment comprises reducing the total number of new gadolinium enhancing T1 lesions as evaluated at weeks 8 and 12, after beginning administration. Thus in some embodiments, provided herein is a method of treating RMS in a subject in need thereof, comprising administering to the subject about 200 mg fenebrutinib twice daily, or an equivalent amount of a pharmaceutically acceptable salt thereof, wherein treating comprises reducing the total number of new gadolinium enhancing T1 lesions at weeks 8 and 12 after beginning administration. In some embodiments, provided herein is a method of reducing the total number of new gadolinium enhancing T1 lesions in a subject with RMS in need thereof, comprising administering to the subject about 200 mg fenebrutinib twice daily, or an equivalent amount of a pharmaceutically acceptable salt thereof, wherein the total number is reduced at weeks 8 and 12 after beginning administration. Further provided is fenebrutinib or a pharmaceutically acceptable salt thereof, for use in treating RMS in a subject in need thereof, comprising administering to the subject about 200 mg fenebrutinib twice daily, or an equivalent amount of a pharmaceutically acceptable salt thereof, wherein the treating comprises reducing the total number of new gadolinium enhancing T1 lesions at weeks 8 and 12 after beginning administration. Still further provided is fenebrutinib or a pharmaceutically acceptable salt thereof for use in reducing the total number of new gadolinium enhancing T1 lesions in a subject with RMS in need thereof, comprising administering to the subject about 200 mg fenebrutinib twice daily, or an equivalent amount of a pharmaceutically acceptable salt thereof, and wherein the total number is reduced at weeks 8 and 12 after beginning administration. In some such embodiments of the methods and uses provided herein, the total number of new gadolinium enhancing T1 lesions at weeks 8 and 12 are reduced by at least 60%. In some embodiments, the reduction is by at least 70%. In some embodiments, the reduction is by at least 80%. In some embodiments, the reduction is by at least 90%. In some embodiments, the total number of new gadolinium enhancing T1 lesions at weeks 8 and 12 is reduced by 92%. In certain embodiments, the reduction is a relative reduction as compared to if no fenebrutinib or pharmaceutically acceptable salt thereof were administered. In some embodiments, fenebrutinib is orally administered. In some embodiments, the subject is orally administered fenebrutinib as two tablets per day wherein each tablet comprises 200 mg fenebrutinib, or as four tablets per day wherein each tablet 100 mg fenebrutinib, for a total daily dose of 400 mg fenebrutinib.

In other embodiments, the treatment comprises reducing by at least 60% the total number of new gadolinium enhancing T1 lesions within 12 weeks after beginning administration. Thus in some embodiments, provided herein is a method of treating RMS in a subject in need thereof, comprising administering to the subject about 200 mg fenebrutinib twice daily, or an equivalent amount of a pharmaceutically acceptable salt thereof, wherein treating comprises reducing by at least 60% the total number of new gadolinium enhancing T1 lesions at 12 weeks after beginning administration of fenebrutinib or an equivalent amount of a pharmaceutically acceptable salt thereof. In some such embodiments, provided herein is a method of reducing the total number of new gadolinium enhancing T1 lesions in a subject in need thereof, comprising administering to the subject about 200 mg fenebrutinib twice daily, or an equivalent amount of a pharmaceutically acceptable salt thereof, and wherein the reduction is at least 60% at week 12 after beginning administration. Further provided is fenebrutinib or a pharmaceutically acceptable salt thereof, for use in treating RMS in a subject in need thereof, comprising administering to the subject about 200 mg fenebrutinib twice daily, or an equivalent amount of a pharmaceutically acceptable salt thereof, wherein the treating comprises reducing by at least 60% the total number of new gadolinium enhancing T1 lesions at 12 weeks after beginning administration. Still further provided is fenebrutinib or a pharmaceutically acceptable salt thereof, for use in reducing the total number of new gadolinium enhancing T1 lesions in a subject with RMS in need thereof, comprising administering to the subject about 200 mg fenebrutinib twice daily, or an equivalent amount of a pharmaceutically acceptable salt thereof, and wherein the total number of new gadolinium enhancing T1 lesions is reduced by at least 60% at 12 weeks after beginning administration. In some such embodiments of the methods and uses provided herein, the total number of new gadolinium enhancing T1 lesions is reduced by at least 70% at week 12. In some embodiments, the reduction is by at least 80% at week 12. In some embodiments, the reduction is 90% at week 12. In certain embodiments, the reduction is a relative reduction as compared to if no fenebrutinib or pharmaceutically acceptable salt thereof were administered. In some embodiments, fenebrutinib is orally administered. In some embodiments, the subject is orally administered fenebrutinib as two tablets per day wherein each tablet comprises 200 mg fenebrutinib, or as four tablets per day wherein each tablet 100 mg fenebrutinib, for a total daily dose of 400 mg fenebrutinib.

In further embodiments, the treatment comprises reducing the rate of developing new gadolinium enhancing T1 lesions. Thus in some embodiments, provided herein is a method of reducing the rate of developing new gadolinium enhancing T1 lesions in a subject with RMS in need thereof, comprising administering to the subject about 200 mg fenebrutinib twice daily, or an equivalent amount of a pharmaceutically acceptable salt thereof. Further provided is fenebrutinib or a pharmaceutically acceptable salt thereof for use in reducing the rate of developing new gadolinium enhancing T1 lesions in a subject with RMS in need thereof, comprising administering to said subject about 200 mg of fenebrutinib or an equivalent amount of a pharmaceutically acceptable salt thereof, twice daily. In certain embodiments of said methods and compound for use, the rate of the development of new gadolinium enhancing T1 lesions is reduced as measured at weeks 4, 8, and 12 after beginning administration. In some embodiments, the rate is reduced at least 40%, for example at least 50%, or at least 60%, or is reduced by 69%. In some embodiments, the rate over 12 weeks after beginning administration is reduced by at least 40%, for example at least 50%, or at least 60%, or is reduced by 69%. In some embodiments, the reduction is compared to the same subject as evaluated immediately prior to beginning administration of fenebrutinib or a pharmaceutically acceptable salt thereof, such as the 12 weeks, 6 months, or 12 months immediately prior. In some embodiments, fenebrutinib is orally administered. In some embodiments, the subject is orally administered fenebrutinib as two tablets daily each comprising 200 mg fenebrutinib, or four tablets daily each comprising 100 mg fenebrutinib, for a total daily dose of 400 mg fenebrutinib. In some embodiments, the rate is an adjusted rate.

Further provided herein is a method of preventing the development of new gadolinium enhancing T1 lesions in a subject with RMS in need thereof, comprising administering to the subject about 200 mg fenebrutinib twice daily, or an equivalent amount of a pharmaceutically acceptable salt thereof. Further provided is fenebrutinib or a pharmaceutically acceptable salt thereof for use in preventing the development of new gadolinium enhancing T1 lesions in a subject with RMS in need thereof, comprising administering to the subject about 200 mg fenebrutinib twice daily, or an equivalent amount of a pharmaceutically acceptable salt thereof. In some embodiments of the methods of, or compound for use in, preventing said development, the development is prevented within 4 weeks after starting administration. In some embodiments, the development is prevented within 8 weeks after starting administration. In some embodiments, the development is prevented within 12 weeks after starting administration. In some embodiments, the prevention of developing new gadolinium enhancing T1 lesions may include a reduction in the number of new gadolinium enhancing T1 lesions as evaluated at 4, 8, or 12 weeks individually; or over 12 weeks as evaluated at 4, 8, or 12 weeks; or at 8 and 12 weeks; as described elsewhere herein (e.g., with a reduction by at least a specified % at a time period as described elsewhere). In some embodiments, fenebrutinib is orally administered. In some embodiments, the subject is orally administered fenebrutinib as two tablets per day wherein each tablet comprises 200 mg fenebrutinib, or as four tablets per day wherein each tablet 100 mg fenebrutinib, for a total daily dose of 400 mg fenebrutinib.

In some embodiments of the methods, compounds for use, and compounds for use in the manufacture of a medicament as provided herein, including the reduction in or prevention of new gadolinium enhancing T1 discussed herein (including as evaluated at time points 4 weeks, 8 weeks, or 12 weeks individually; or over 12 weeks as evaluated at 4 weeks, 8 weeks, or 12 weeks; or evaluated at 8 and 12 weeks; or any other combination; or as a rate reduction), the comparison is relative to a subject who is not administered fenebrutinib, or a pharmaceutically acceptable salt thereof. In some embodiments, this comparison subject is the same subject, but before beginning administration of fenebrutinib or a pharmaceutically acceptable salt thereof, for example as evaluated in the same subject in the immediate 4, weeks, immediate 8 weeks, immediate 12 weeks, immediate 6 months, or immediate 12 months before beginning administration of fenebrutinib or a pharmaceutically acceptable salt thereof. In some embodiments, this comparison subject is another subject with RMS who is not administered fenebrutinib or pharmaceutically acceptable salt thereof, such as another subject with RMS who is not administered any RMS therapy.

In certain embodiments of the methods, compound for use, and compound for use in the manufacture of a medicament as provided herein, the reduction in or prevention of developing new gadolinium enhancing T1 lesions (including as evaluated at time points 4 weeks, 8 weeks, or 12 weeks individually; or over 12 weeks as evaluated at 4 weeks, 8 weeks, or 12 weeks; or evaluated at 8 and 12 weeks; or any other combination; or as a rate reduction) is clinically illustrated by comparing a subject (or group of subjects) who was administered fenebrutinib or pharmaceutically acceptable salt thereof, to a subject (or group of subjects) who was not administered an RMS therapy (e.g., a non-active comparator, or placebo). In some embodiments, the comparison is relative to the same subject before beginning administration of fenebrutinib or a pharmaceutically acceptable salt thereof, for example as evaluated in the same subject in the immediate 12 weeks, immediate 6 months, or immediate 12 months before beginning administration of fenebrutinib or a pharmaceutically acceptable salt thereof. In some embodiments, the comparison is relative to another subject that is not administered an RMS therapy. Based on this data, a single individual would be generally expected to experience a relative reduction in or prevention of developing said new gadolinium enhancing T1 lesions when taking fenebrutinib or a pharmaceutically acceptable salt thereof, compared to if that same individual were not taking an RMS therapy. In some embodiments, the reduction is a reduction in the rate of developing new lesions. In some embodiments, the rate is an adjusted rate.

### B. New or Enlarging T2-Weighted Lesions

Provided herein are methods of treating RMS in a subject in need thereof, wherein the treatment comprises reducing the number of new or enlarging T2-weighted lesions over time, or reducing the rate of generation of new or enlarging T2-weighted lesions. Similarly, provided herein is fenebrutinib or a pharmaceutically acceptable salt thereof for use in treating RMS in a subject in need thereof, wherein the treatment comprises reducing the number of new or enlarging T2-weighted lesions over time, or reducing the rate of generation of new or enlarging T2-weighted lesions. In some embodiments, the subject is administered fenebrutinib. In some embodiments, the subject is orally administered 200 mg fenebrutinib twice daily, comprising a total daily dose of 400 mg.

In some embodiments of the methods and compound for use provided herein, the treatment comprises reducing the total number of new or enlarging T2-weighted lesions over 12 weeks after beginning administration of fenebrutinib or an equivalent amount of a pharmaceutically acceptable salt thereof, as evaluated at 4, 8, and 12 weeks. Thus in some embodiments, provided herein is a method of treating RMS in a subject in need thereof, comprising administering to the subject about 200 mg fenebrutinib twice daily, or an equivalent amount of a pharmaceutically acceptable salt thereof, wherein the treating comprises reducing the total number of new or enlarging T2-weighted lesions over 12 weeks after beginning administration, as evaluated at 4, 8, and 12 weeks. Also provided herein is a method of reducing the total number of new or enlarging T2-weighted lesions in a subject with RMS in need thereof, comprising administering to the subject about 200 mg fenebrutinib twice daily, or an equivalent amount of a pharmaceutically acceptable salt thereof, wherein the total number is reduced over 12 weeks after beginning administration, as evaluated at 4, 8, and 12 weeks. Further provided is fenebrutinib or a pharmaceutically acceptable salt thereof, for use in treating RMS in a subject in need thereof, comprising administering to the subject about 200 mg fenebrutinib twice daily, or an equivalent amount of a pharmaceutically acceptable salt thereof, wherein the treating comprises reducing the total number of new or enlarging T2-weighted lesions over 12 weeks after beginning administration, as evaluated at 4, 8, and 12 weeks. Also provided is fenebrutinib or a pharmaceutically acceptable salt thereof for use in reducing the total number of new or enlarging T2-weighted lesions in a subject with RMS in need thereof, comprising administering to the subject about 200 mg fenebrutinib twice daily, or an equivalent amount of a pharmaceutically acceptable salt thereof, and wherein the total number is reduced over 12 weeks after beginning administration, as evaluated at 4, 8, and 12 weeks. In some such embodiments of the methods and uses provided herein, the total number of new or enlarging T2-weighted lesions over 12 weeks after beginning administration, as evaluated at 4, 8, and 12 weeks, is reduced by at least 50%. In some embodiments, the reduction is by at least 60%. In certain embodiments, the reduction is by at least 70%. In some embodiments, the reduction is by 73.5%. In certain embodiments, the reduction is a relative reduction as compared to if no fenebrutinib or pharmaceutically acceptable salt thereof were administered. In some embodiments, fenebrutinib is orally administered. In some embodiments, the subject is orally administered fenebrutinib as two tablets per day wherein each tablet comprises 200 mg fenebrutinib, or as four tablets per day wherein each tablet 100 mg fenebrutinib, for a total daily dose of 400 mg fenebrutinib.

In other embodiments, the treatment comprises reducing the total number of new or enlarging T2-weighted lesions at 4 weeks after beginning administration of fenebrutinib or an equivalent amount of a pharmaceutically acceptable salt thereof. Thus in some embodiments, provided herein is a method of treating RMS in a subject in need thereof, comprising administering to the subject about 200 mg fenebrutinib twice daily, or an equivalent amount of a pharmaceutically acceptable salt thereof, wherein treating comprises reducing the total number of new or enlarging T2-weighted lesions at 4 weeks after beginning administration. In some such embodiments, provided herein is a method of reducing the total number of new or enlarging T2-weighted lesions in a subject with RMS in need thereof, comprising administering to the subject about 200 mg fenebrutinib twice daily, or an equivalent amount of a pharmaceutically acceptable salt thereof, wherein the total number is reduced at 4 weeks after beginning administration. Further provided is fenebrutinib or a pharmaceutically acceptable salt thereof, for use in treating RMS in a subject in need thereof, comprising administering to the subject about 200 mg fenebrutinib twice daily, or an equivalent amount of a pharmaceutically acceptable salt thereof, wherein the treating comprises reducing the total number of new or enlarging T2-weighted lesions at 4 weeks after beginning administration. Also provided is fenebrutinib or a pharmaceutically acceptable salt thereof for use in reducing the total number of new or enlarging T2-weighted lesions in a subject with RMS in need thereof, comprising administering to the subject about 200 mg fenebrutinib twice daily, or an equivalent amount of a pharmaceutically acceptable salt thereof, and wherein the total number is reduced at 4 weeks after beginning administration. In some such embodiments of the methods and uses provided herein, the total number of new or enlarging T2-weighted lesions are reduced by at least 30% at 4 weeks. In some embodiments, the reduction is by at least 40% at 4 weeks. In some embodiments, the reduction is by 49% at 4 weeks. In certain embodiments, the reduction is a relative reduction as compared to if no fenebrutinib or pharmaceutically acceptable salt thereof were administered. In some embodiments, fenebrutinib is orally administered. In some embodiments, the subject is orally administered fenebrutinib as two tablets per day wherein each tablet comprises 200 mg fenebrutinib, or as four tablets per day wherein each tablet 100 mg fenebrutinib, for a total daily dose of 400 mg fenebrutinib.

In still further embodiments, the treatment comprises reducing the total number of new or enlarging T2-weighted lesions at 8 weeks after beginning administration of fenebrutinib or an equivalent amount of a pharmaceutically acceptable salt thereof. Thus in some embodiments, provided herein is a method of treating RMS in a subject in need thereof, comprising administering to the subject about 200 mg fenebrutinib twice daily, or an equivalent amount of a pharmaceutically acceptable salt thereof, wherein treating comprises reducing the total number of new or enlarging T2-weighted lesions at 8 weeks after beginning administration. In some such embodiments, provided herein is a method of reducing the total number of new or enlarging T2-weighted lesions in a subject with RMS in need thereof, comprising administering to the subject about 200 mg fenebrutinib twice daily, or an equivalent amount of a pharmaceutically acceptable salt thereof, wherein the total number is reduced at 8 weeks after beginning administration. Further provided is fenebrutinib or a pharmaceutically acceptable salt thereof, for use in treating RMS in a subject in need thereof, comprising administering to the subject about 200 mg fenebrutinib twice daily, or an equivalent amount of a pharmaceutically acceptable salt thereof, wherein the treating comprises reducing the total number of new or enlarging T2-weighted lesions at 8 weeks after beginning administration. Also provided is fenebrutinib or a pharmaceutically acceptable salt thereof for use in reducing the total number of new or enlarging T2-weighted lesions in a subject with RMS in need thereof, comprising administering to the subject about 200 mg fenebrutinib twice daily, or an equivalent amount of a pharmaceutically acceptable salt thereof, and wherein the total number is reduced at 8 weeks after beginning administration. In some such embodiments of the methods and uses provided herein, the total number of new or enlarging T2-weighted lesions is reduced by at least 60% at 8 weeks. In some embodiments, the reduction is by at least 70% at 8 weeks. In some embodiments, the reduction is by at least 80% at 8 weeks. In some embodiments, the reduction is by at least 90% at 8 weeks. In certain embodiments, the reduction is a relative reduction as compared to if no fenebrutinib or pharmaceutically acceptable salt thereof were administered. In some embodiments, the subject is orally administered fenebrutinib as two tablets per day wherein each tablet comprises 200 mg fenebrutinib, or as four tablets per day wherein each tablet 100 mg fenebrutinib, for a total daily dose of 400 mg fenebrutinib.

In yet further embodiments, the treatment comprises reducing the total number of new or enlarging T2-weighted lesions as evaluated at weeks 8 and 12, after beginning administration of fenebrutinib or an equivalent amount of a pharmaceutically acceptable salt thereof. Thus in some embodiments, provided herein is a method of treating RMS in a subject in need thereof, comprising administering to the subject about 200 mg fenebrutinib twice daily, or an equivalent amount of a pharmaceutically acceptable salt thereof, and wherein the treating comprises reducing the total number of new or enlarging T2-weighted lesions at weeks 8 and 12 after beginning administration of fenebrutinib or an equivalent amount of a pharmaceutically acceptable salt thereof. In some such embodiments, provided herein is a method of reducing the total number of new or enlarging T2-weighted lesions at weeks 8 and 12 after beginning administration of fenebrutinib or an equivalent amount of a pharmaceutically acceptable salt thereof. In some such embodiments, the new or enlarging T2-weighted lesions at weeks 8 and 12 are reduced by at least 60%. In some embodiments, the reduction is by at least 70%. In some embodiments, the reduction is by at least 80%. In some embodiments, the reduction is by at least 90%. In some embodiments, the reduction is by 93%. In certain embodiments, the reduction is a relative reduction as compared to if no fenebrutinib or pharmaceutically acceptable salt thereof were administered. In some embodiments, fenebrutinib is orally administered. In some embodiments, the subject is orally administered fenebrutinib as two tablets per day wherein each tablet comprises 200 mg fenebrutinib, or as four tablets per day wherein each tablet 100 mg fenebrutinib, for a total daily dose of 400 mg fenebrutinib.

In other embodiments, the treatment comprises reducing by at least 60% the total number of new or enlarging T2-weighted lesions at 12 weeks after beginning administration of fenebrutinib or an equivalent amount of a pharmaceutically acceptable salt thereof. Thus in some embodiments, provided herein is a method of treating RMS in a subject in need thereof, comprising administering to the subject about 200 mg fenebrutinib twice daily, or an equivalent amount of a pharmaceutically acceptable salt thereof, wherein treating comprises reducing by at least 60% the total number of new or enlarging T2-weighted lesions at 12 weeks after beginning administration. In some such embodiments, provided herein is a method of reducing the total number of new or enlarging T2-weighted lesions in a subject with RMS in need thereof, comprising administering to the subject about 200 mg fenebrutinib twice daily, or an equivalent amount of a pharmaceutically acceptable salt thereof, and wherein the reduction is at least 60% at 12 weeks after beginning administration. Further provided is fenebrutinib or a pharmaceutically acceptable salt thereof, for use in treating RMS in a subject in need thereof, comprising administering to the subject about 200 mg fenebrutinib twice daily, or an equivalent amount of a pharmaceutically acceptable salt thereof, wherein the treating comprises reducing by at least 60% the total number of new or enlarging T2-weighted lesions at 12 weeks after beginning administration. Still further provided is fenebrutinib or a pharmaceutically acceptable salt thereof, for use in reducing the total number of new or enlarging T2-weighted lesions in a subject with RMS in need thereof, comprising administering to the subject about 200 mg fenebrutinib twice daily, or an equivalent amount of a pharmaceutically acceptable salt thereof, and wherein the total number of new or enlarging T2-weighted lesions is reduced by at least 60% at 12 weeks after beginning administration. In some such embodiments of the methods and uses provided herein, the total number of new or enlarging T2-weighted lesions are reduced by at least 70% at 12 weeks. In some embodiments, reduction is by at least 80% at 12 weeks. In some embodiments, the reduction is by at least 90% at 12 weeks. In still further embodiments, reduction is by 95% at 12 weeks. In certain embodiments, the reduction is a relative reduction as compared to if no fenebrutinib or pharmaceutically acceptable salt thereof were administered. In some embodiments, fenebrutinib is orally administered. In some embodiments, the subject is orally administered fenebrutinib as two tablets per day wherein each tablet comprises 200 mg fenebrutinib, or as four tablets per day wherein each tablet 100 mg fenebrutinib, for a total daily dose of 400 mg fenebrutinib.

In further embodiments, the treatment comprises reducing the rate of developing new or enlarging T2-weighted lesions. Thus in some embodiments, provided herein is a method of reducing the rate of developing new or enlarging T2-weighted lesions in a subject with RMS in need thereof, comprising administering to the subject about 200 mg fenebrutinib twice daily, or an equivalent amount of a pharmaceutically acceptable salt thereof. Further provided is fenebrutinib or a pharmaceutically acceptable salt thereof for use in reducing the rate of developing new or enlarging T2-weighted lesions in a subject with RMS in need thereof, comprising administering to said subject about 200 mg of fenebrutinib or an equivalent amount of a pharmaceutically acceptable salt thereof, twice daily. In certain embodiments of said methods and compounds for use, the rate of developing new or enlarging T2-weighted lesions is reduced as measured at weeks 4, 8, and 12 after beginning administration. In some embodiments, the rate is reduced at least 40%, for example at least 50%, or at least 60%, or is reduced by at least 70%, or is reduced by 74%. In some embodiments, the rate over 12 weeks after beginning administration is reduced by at least 40%, for example at least 50%, or at least 60%, or is reduced by at least 70%, or is reduced 74%. In some embodiments, the reduction is compared to the same subject as evaluated immediately prior to beginning administration of fenebrutinib or a pharmaceutically acceptable salt thereof, such as the 12 weeks, 6 months, or 12 months immediately prior. In some embodiments, the reduction is compared to another subject with RMS who is not administered fenebrutinib or a pharmaceutically acceptable salt thereof, such as a subject who is not administered any RMS therapy. In some embodiments, fenebrutinib is orally administered. In some embodiments, the subject is orally administered fenebrutinib as two tablets daily each comprising 200 mg fenebrutinib, or four tablets daily each comprising 100 mg fenebrutinib, for a total daily dose of 400 mg fenebrutinib. In some embodiments, the rate is an adjusted rate.

Further provided herein is a method of preventing the development of new or enlarging T2-weighted lesions in a subject with RMS in need thereof, comprising administering to the subject about 200 mg fenebrutinib twice daily, or an equivalent amount of a pharmaceutically acceptable salt thereof. Further provided is fenebrutinib or a pharmaceutically acceptable salt thereof for use in preventing the development of new or enlarging T2-weighted lesions in a subject with RMS in need thereof, comprising administering to the subject about 200 mg fenebrutinib twice daily, or an equivalent amount of a pharmaceutically acceptable salt thereof. In some embodiments of the methods of or compound for use in preventing said development, the development is prevented within 4 weeks after starting administration. In some embodiments, the development is prevented within 8 weeks after starting administration. In some embodiments, the development is prevented within 12 weeks after starting administration. In some embodiments, the prevention of developing new or enlarging T2-weighted lesions may include a reduction in the number of new or enlarging T2-weighted lesions as evaluated at 4, 8, or 12 weeks individually; or over 12 weeks as evaluated at 4, 8, or 12 weeks; or at 8 and 12 weeks; as described elsewhere herein (e.g., with a reduction by at least a specified % at a time period as described elsewhere). In some embodiments, fenebrutinib is orally administered. In some embodiments, the subject is orally administered fenebrutinib as two tablets daily each comprising 200 mg fenebrutinib, or four tablets daily each comprising 100 mg fenebrutinib, for a total daily dose of 400 mg fenebrutinib.

In some embodiments of the methods, compounds for use, and compounds for use in the manufacture of a medicament as provided herein, including the reduction in or prevention of developing new or enlarging T2-weighted lesions discussed herein (including as evaluated at time points 4 weeks, 8 weeks, or 12 weeks individually; or over 12 weeks as evaluated at 4 weeks, 8 weeks, or 12 weeks; or evaluated at 8 and 12 weeks; or any other combination; or as a rate reduction), the comparison is relative to a subject who is not administered fenebrutinib, or a pharmaceutically acceptable salt thereof. In some embodiments, this comparison subject is the same subject, but before beginning administration of fenebrutinib or a pharmaceutically acceptable salt thereof, for example as evaluated in the same subject in the immediate 4, weeks, immediate 8 weeks, immediate 12 weeks, immediate 6 months, or immediate 12 months before beginning administration of fenebrutinib or a pharmaceutically acceptable salt thereof. In some embodiments, this comparison subject is another subject with RMS who is not administered fenebrutinib or pharmaceutically acceptable salt thereof, such as another subject with RMS who is not administered any RMS therapy.

In certain embodiments of the methods, compounds for use, and compounds for use in the manufacture of a medicament as provided herein, the reduction in or prevention of developing new or enlarging T2-weighted lesions discussed (including as evaluated at time points 4 weeks, 8 weeks, or 12 weeks individually; or over 12 weeks as evaluated at 4 weeks, 8 weeks, or 12 weeks; or evaluated at 8 and 12 weeks; or any other combination; or as a rate reduction) is clinically illustrated by comparing a subject (or group of subjects) who was administered fenebrutinib or pharmaceutically acceptable salt thereof, to a subject (or group of subjects) who was not administered an RMS therapy (e.g., a non-active comparator, or placebo). Based on this data, a single individual would be generally expected to experience a relative reduction in or prevention of developing said new or enlarging T2-weighted lesions when taking fenebrutinib or a pharmaceutically acceptable salt thereof, compared to if that same individual were not taking an RMS therapy. In some embodiments, the reduction is a reduction in the rate of development. In some embodiments, the rate is an adjusted rate.

### C. Odds of Both New Gadolinium Enhancing T1 Lesions and New or Enlarging T2-Weighted Lesions

Provided herein are methods of treating RMS in a subject in need thereof, comprising administering to the subject about 200 mg fenebrutinib twice daily, or an equivalent amount of a pharmaceutically acceptable salt thereof, and wherein the treatment comprises increasing the odds that a subject will be free from both (a) any new gadolinium enhancing T1 lesions; and (b) any new or enlarging T2-weighted lesions. Similarly, provided herein is fenebrutinib or a pharmaceutically acceptable salt thereof for use in treating RMS in a subject in need thereof, comprising administering to the subject about 200 mg fenebrutinib twice daily, or an equivalent amount of a pharmaceutically acceptable salt thereof, wherein treatment comprises increase the odds that a subject will be free from both (a) any new gadolinium enhancing T1 lesions; and (b) any new or enlarging T2-weighted lesions. In still further embodiments, provided herein is a method of increasing the odds that a subject with RMS will be free from both (a) any new gadolinium enhancing T1 lesions; and (b) any new or enlarging T2-weighted lesions, comprising administering to the subject about 200 mg fenebrutinib twice daily, or an equivalent amount of a pharmaceutically acceptable salt thereof. Still further, provided herein is fenebrutinib or a pharmaceutically acceptable salt thereof for increasing the odds that a subject with RMS will be free from both (a) any new gadolinium enhancing T1 lesions; and (b) any new or enlarging T2-weighted lesions, comprising administering to the subject about 200 mg fenebrutinib twice daily, or an equivalent amount of a pharmaceutically acceptable salt thereof. In some embodiments of the methods and uses provided herein for increasing the odds of being free from new lesions, the subject is at least two times more likely to be free from both (a) any new gadolinium enhancing T1 lesions; and (b) any new or enlarging T2-weighted lesions. In some embodiments, the subject is at least three times more likely to be free from both (a) any new gadolinium enhancing T1 lesions; and (b) any new or enlarging T2-weighted lesions. In some embodiments, the subject is four times more likely to not develop both (a) any new gadolinium enhancing T1 lesions; and (b) any new or enlarging T2-weighted lesions. Increasing the odds “to be free from” may also be expressed as increasing the odds “to not develop”. In some embodiments, this increase in the odds of being free from, or not developing, both types of lesions is over a particular time period. For example, in some embodiments, the odds of being free from developing new lesions as described is over the first 12 weeks after beginning administration, as measured at weeks 4, 8, and 12. Thus, for example, provided herein is fenebrutinib or a pharmaceutically acceptable salt thereof for use in, or a method of, increasing the odds that a subject will be free from both (a) any new gadolinium enhancing T1 lesions; and (b) any new or enlarging T2-weighted lesions over the first 12 weeks after beginning administration of fenebrutinib or a pharmaceutically acceptable salt thereof, wherein the increase in odds is at least 2 fold. In some embodiments, the increase is at least 3 fold, over the first 12 weeks after beginning administration. In some embodiments the increase is at least 4 fold, over the first 12 weeks after beginning administration. In some embodiments, fenebrutinib is orally administered. In some embodiments, the subject is orally administered fenebrutinib as two tablets daily each comprising 200 mg fenebrutinib, or four tablets daily each comprising 100 mg fenebrutinib, for a total daily dose of 400 mg fenebrutinib. In some embodiments of the methods, compounds for use, and compounds for use in the manufacture of a medicament as provided herein, including increasing the odds that a subject will be free from both (a) any new gadolinium enhancing T1 lesions; and (b) any new or enlarging T2-weighted lesions (including as evaluated at time points 4 weeks, 8 weeks, or 12 weeks individually; or over 12 weeks as evaluated at 4 weeks, 8 weeks, or 12 weeks; or evaluated at 8 and 12 weeks; or any other combination), the comparison is relative to a subject who is not administered fenebrutinib, or a pharmaceutically acceptable salt thereof. In some embodiments, this comparison subject is the same subject, but before beginning administration of fenebrutinib or a pharmaceutically acceptable salt thereof, for example as evaluated in the same subject in the immediate 4, weeks, immediate 8 weeks, immediate 12 weeks, immediate 6 months, or immediate 12 months before beginning administration of fenebrutinib or a pharmaceutically acceptable salt thereof. In some embodiments, this comparison subject is another subject with RMS who is not administered fenebrutinib or pharmaceutically acceptable salt thereof, such as another subject with RMS who is not administered any RMS therapy.

### D. T1 Hypointense Lesions

Provided herein are methods of treating RMS in a subject in need thereof, wherein the treatment comprises reducing the number of new T1 hypointense lesions over time, or reducing the rate of generation of new T1 hypointense lesions. Similarly, provided herein is fenebrutinib or a pharmaceutically acceptable salt thereof for use in treating RMS in a subject in need thereof, wherein the treatment comprises reducing the number of new T1 hypointense lesions over time, or reducing the rate of generation of new T1 hypointense lesions. In some embodiments, the subject is administered fenebrutinib. In some embodiments, the subject is orally administered 200 mg fenebrutinib twice daily, comprising a total daily dose of 400 mg. In some embodiments, the subject is a human.

In some embodiments of the methods and compound for use provided herein, the treatment comprises reducing the total number of new T1 hypointense lesions after beginning administration of fenebrutinib or an equivalent amount of a pharmaceutically acceptable salt thereof. Thus in some embodiments, provided herein is a method of treating RMS in a subject in need thereof, comprising administering to the subject about 200 mg fenebrutinib twice daily, or an equivalent amount of a pharmaceutically acceptable salt thereof, wherein the treating comprises reducing the total number of new T1 hypointense lesions after beginning administration. Further provided is fenebrutinib or a pharmaceutically acceptable salt thereof, for use in treating RMS in a subject in need thereof, comprising administering to the subject about 200 mg fenebrutinib twice daily, or an equivalent amount of a pharmaceutically acceptable salt thereof, wherein the treating comprises reducing the total number of new T1 hypointense lesions. In some embodiments of the methods and compound for use provided, the reduction is over 12 weeks after beginning administration. In some embodiments, the reduction is over 4 weeks. In some embodiments, the reduction is over 8 weeks. In some embodiments, the reduction is over 12 weeks, as evaluated at 4, 8, and 12 weeks.

In some such embodiments of the methods and uses provided herein, the total number of new T1 hypointense lesions is reduced by at least 40%. In some embodiments, the reduction is by at least 50%. In certain embodiments, the reduction is by at least 60%. In some embodiments, the reduction is by 64%. In some embodiments, the reduction is over 12 weeks, and is at least 40%, for example at least 50%, such as at least 55%, or is 58%. In some embodiments, the reduction is over 8 weeks, and is at least 40%, for example at least 50%, or at least 60%, or is 64%. In some embodiments, the reduction is over 4 weeks, and is at least 30%, for example at least 40%, or at least 45%, or is 48%. In some embodiments, the combined reduction at 4, 8, and 12 weeks is at least 40%, for example at least 50%, or at least 60%, or is 64%. In certain embodiments, the reduction is a relative reduction as compared to if no fenebrutinib or pharmaceutically acceptable salt thereof were administered. In some embodiments, the reduction is compared to the same subject as evaluated immediately prior to beginning administration of fenebrutinib or a pharmaceutically acceptable salt thereof, such as the 12 weeks, 6 months, or 12 months immediately prior. In some embodiments, fenebrutinib is orally administered. In some embodiments, the subject is orally administered fenebrutinib as two tablets per day wherein each tablet comprises 200 mg fenebrutinib, or as four tablets per day wherein each tablet 100 mg fenebrutinib, for a total daily dose of 400 mg fenebrutinib. In some embodiments, the subject is a human.

In further embodiments, the treatment comprises reducing the rate of developing new T1 hypointense lesions. Thus in some embodiments, provided herein is a method of reducing the rate of developing new T1 hypointense lesions in a subject with RMS in need thereof, comprising administering to the subject about 200 mg fenebrutinib twice daily, or an equivalent amount of a pharmaceutically acceptable salt thereof. Further provided is fenebrutinib or a pharmaceutically acceptable salt thereof for use in reducing the rate of developing new T1 hypointense lesions in a subject with RMS in need thereof, comprising administering to said subject about 200 mg of fenebrutinib or an equivalent amount of a pharmaceutically acceptable salt thereof, twice daily. In certain embodiments of said methods and compounds for use, the rate of developing new T1 hypointense lesions is reduced as measured at weeks 4, 8, and 12 after beginning administration. In some embodiments, the rate is measured at 12 weeks after beginning administration. In some embodiments, the rate is measured at 8 weeks after beginning administration. In some embodiments, the rate is measured at 4 weeks after beginning administration. In some embodiments, the rate is over 12 weeks after beginning administration, as measured at 4, 8, and 12 weeks. In some embodiments, the rate is reduced at least 40%, for example at least 50%, or at least 60%, or is reduced by 64%. In some embodiments, the rate over 12 weeks after beginning administration is reduced by at least 40%, for example at least 50%, or at least 60%, or is reduced by 64%. In some embodiments, the reduction is compared to the same subject as evaluated immediately prior to beginning administration of fenebrutinib or a pharmaceutically acceptable salt thereof, such as the 12 weeks, 6 months, or 12 months immediately prior. In some embodiments, fenebrutinib is orally administered. In some embodiments, the subject is orally administered fenebrutinib as two tablets daily each comprising 200 mg fenebrutinib, or four tablets daily each comprising 100 mg fenebrutinib, for a total daily dose of 400 mg fenebrutinib. In some embodiments, the subject is a human. In some embodiments, the rate is an adjusted rate.

Further provided herein is a method of preventing the development of new T1 hypointense lesions in a subject with RMS in need thereof, comprising administering to the subject about 200 mg fenebrutinib twice daily, or an equivalent amount of a pharmaceutically acceptable salt thereof. Further provided is fenebrutinib or a pharmaceutically acceptable salt thereof for use in preventing the development of new T1 hypointense lesions in a subject with RMS in need thereof, comprising administering to the subject about 200 mg fenebrutinib twice daily, or an equivalent amount of a pharmaceutically acceptable salt thereof. In some embodiments of the methods of or compound for use in preventing said development, the development is prevented within 4 weeks after starting administration. In some embodiments, the development is prevented within 8 weeks after starting administration. In some embodiments, the development is prevented within 12 weeks after starting administration. In some embodiments, the prevention of developing new T1 hypointense lesions may include a reduction in the number of new T1 hypointense lesions as evaluated at 4, 8, or 12 weeks individually; or over 12 weeks as evaluated at 4, 8, or 12 weeks; or at 8 and 12 weeks; as described elsewhere herein (e.g., with a reduction by at least a specified % at a time period as described elsewhere). In some embodiments, the prevention is compared to the same subject as evaluated immediately prior to beginning administration of fenebrutinib or a pharmaceutically acceptable salt thereof, such as the 12 weeks, 6 months, or 12 months immediately prior. In some embodiments, the subject is human. In some embodiments, fenebrutinib is orally administered. In some embodiments, the subject is orally administered fenebrutinib as two tablets daily each comprising 200 mg fenebrutinib, or four tablets daily each comprising 100 mg fenebrutinib, for a total daily dose of 400 mg fenebrutinib.

In some embodiments of the methods, compounds for use, and compounds for use in the manufacture of a medicament as provided herein, including the reduction in or prevention of developing new gadolinium enhancing T1 lesions (including as evaluated at time points 4 weeks, 8 weeks, or 12 weeks individually; or over 12 weeks as evaluated at 4 weeks, 8 weeks, or 12 weeks; or evaluated at 8 and 12 weeks; or the rate; or any other combination), the comparison is relative to a subject who is not administered fenebrutinib, or a pharmaceutically acceptable salt thereof. In some embodiments, this comparison subject is the same subject, but before beginning administration of fenebrutinib or a pharmaceutically acceptable salt thereof, for example as evaluated in the same subject in the immediate 4, weeks, immediate 8 weeks, immediate 12 weeks, immediate 6 months, or immediate 12 months before beginning administration of fenebrutinib or a pharmaceutically acceptable salt thereof. In some embodiments, this comparison subject is another subject with RMS who is not administered fenebrutinib or pharmaceutically acceptable salt thereof, such as another subject with RMS who is not administered any RMS therapy.

In certain embodiments of the methods, compounds for use, and compounds for use in the manufacture of a medicament as provided herein, the reduction in or prevention of developing new T1 hypointense lesions discussed (including as evaluated at time points 4 weeks, 8 weeks, or 12 weeks individually; or over 12 weeks as evaluated at 4 weeks, 8 weeks, or 12 weeks; or evaluated at 8 and 12 weeks; or any other combination; or as a rate reduction) is clinically illustrated by comparing a subject (or group of subjects) who was administered fenebrutinib or pharmaceutically acceptable salt thereof, to a subject (or group of subjects) who was not administered an RMS therapy (e.g., a non-active comparator, or placebo). In some embodiments, the comparison is relative to the same subject before beginning administration of fenebrutinib or a pharmaceutically acceptable salt thereof, for example as evaluated in the same subject in the immediate 12 weeks, immediate 6 months, or immediate 12 months before beginning administration of fenebrutinib or a pharmaceutically acceptable salt thereof. In some embodiments, the comparison is relative to another subject that is not administered an RMS therapy. Based on this data, a single individual would be generally expected to experience a relative reduction in or prevention of developing said new T1 hypointense lesions when taking fenebrutinib or a pharmaceutically acceptable salt thereof, compared to if that same individual were not taking an RMS therapy. In some embodiments, the reduction is a reduction in the rate of development. In some embodiments, the rate is an adjusted rate.

### E. Cerebrospinal Fluid Concentration

Provided herein are methods of treating MS in a subject in need thereof, wherein the method comprises orally administering to the subject a sufficient amount of a BTK inhibitor to reach a cerebrospinal fluid (CSF) concentration greater than the active inhibitory threshold for inhibition of B-cell activation, inhibition of myeloid lineage cell activation, inhibition of BTK, or any combination thereof, for said BTK inhibitor. Similarly, provided herein is a BTK inhibitor for use in treating MS in a subject in need thereof, wherein the treatment comprises orally administering to the subject a sufficient amount of a BTK inhibitor to reach a CSF concentration greater than the active inhibitory threshold for inhibition of B-cell activation, inhibition of myeloid lineage cell activation, inhibition of BTK, any combination thereof, for said BTK inhibitor. In some embodiments, the MS is RMS. In some embodiments, the MS is PPMS. In some embodiments, the BTK inhibitor is fenebrutinib or a pharmaceutically acceptable salt thereof. In some embodiments, the subject is orally administered 200 mg fenebrutinib twice daily, comprising a total daily dose of 400 mg, or an equivalent amount of a pharmaceutically acceptable salt thereof. In some embodiments, the subject is orally administered fenebrutinib. In some embodiments, the subject is a human.

In certain embodiments of the methods and uses provided herein, the CSF concentration of the BTK inhibitor is greater than the active inhibitory threshold for inhibition of B-cell activation. In some embodiments, the CSF concentration of the BTK inhibitor is greater than the maximal inhibitory threshold for inhibition of B-cell activation. In some embodiments, the active inhibitory threshold, or maximal inhibitory threshold, or both, for inhibiting B-cell activation is determined by an in vitro cell assay. In some such embodiments, an in vitro CD69 expression assay is used, such as the anti-IgM-induced expression of CD69 on B cells. CD69 is a B-cell activation marker which may be used to evaluate the inhibition of B-cell activation. In some embodiments, the active inhibitory threshold is the IC₅₀ in such an assay, and the maximal inhibitory threshold is the IC₉₀ in such an assay. In certain embodiments, the anti-IgM-induced expression of CD69 on B cells is measured in a whole human blood assay. The methods for performing such an assay are known to those of skill in the art, and may be found, for example in Crawford J J, et al. J Med Chem 2018; 6:2227-2245. In some embodiments, the active inhibitory threshold is the IC₅₀, or the maximal inhibitory threshold is the IC₉₀, or both, for inhibiting B-cell activation as determined by an in vitro cell assay evaluating anti-IgM-induced expression of CD69 on B cells in a whole human blood assay; wherein the assay is run with at a minimum of three separate blood donors, such as between three to ten blood donors, for example four, five, six, seven, eight, nine, or ten blood donors; and the IC₅₀, or IC₉₀, or both, as applicable is the mean of multiple trials. In some embodiments, the subject is orally administered a sufficient amount of a BTK inhibitor to reach a CSF concentration greater than 20% below the maximal inhibitory threshold for inhibition of B-cell activation. For example, if the maximal inhibitory threshold were 100 ng/mL, 20% below said threshold is 80 ng/mL, and the CSF concentration would be greater than 80 ng/mL. In some embodiments, the subject is orally administered a sufficient amount of a BTK inhibitor to reach a CSF concentration between 20% to 250% of the maximal inhibitory threshold for inhibition of B-cell activation. For example if the maximal inhibitory threshold were 100 ng/mL, between 20% to 250% of said threshold would be between 20 ng/mL and 250 ng/mL. In some embodiments, the subject is orally administered a sufficient amount of a BTK inhibitor to reach a CSF concentration between 20% to 225%, between 25% to 200%, between 30% to 190%, or between 40% to 170% of the maximal inhibitory threshold for inhibition of B-cell activation. In some embodiments, the BTK inhibitor is fenebrutinib or a pharmaceutically acceptable salt thereof, and the subject is orally administered a sufficient amount of a BTK inhibitor to reach a CSF concentration of at least 11 ng/mL. In some embodiments, the subject is orally administered a sufficient amount of a BTK inhibitor to reach a CSF concentration of at least 15 ng/ml, or at least 20 ng/mL, or at least 30 ng/mL, or at least 33 ng/mL, or at least 43.5 ng/mL. In some embodiments, the subject is orally administered a sufficient amount of a BTK inhibitor to reach a CSF concentration between 11 ng/ml to 80 ng/mL, or between 15 ng/mL and 80 ng/mL, or between 30 ng/ml and 80 ng/mL, or between 33 ng/ml and 75 ng/mL. In some embodiments, the CSF concentration is reached at least 12 weeks after beginning oral administration of the BTK inhibitor. In some embodiments, the CSF concentration is reached at least 8 weeks after beginning oral administration of the BTK inhibitor. In some embodiments, the CSF concentration is reached at least 4 weeks after beginning oral administration of the BTK inhibitor.

In certain embodiments of the methods and uses provided herein, the CSF concentration of the BTK inhibitor is greater than the active inhibitory threshold for inhibition of myeloid lineage cell activation. In some embodiments, the CSF concentration of the BTK inhibitor is greater than the maximal inhibitory threshold for inhibition of myeloid lineage cell activation. In some embodiments, the myeloid lineage cell is basophils. In some embodiments, the active inhibitory threshold, or maximal inhibitory threshold, or both, for inhibiting myeloid lineage cell activation is determined by an in vitro cell assay. In some such embodiments, an in vitro assay measuring the expression of a cell surface marker of a myeloid lineage cell is used, for example the expression of a cell surface marker of basophils, such as the expression of CD63, for example through FcεR mediated basophil activation as assessed by surface expression of CD63. CD63 is a marker the cell surface expression of which may be used to evaluate the inhibition of myeloid lineage activation, for example basophil activation. In some embodiments, the active inhibitory threshold is the IC₅₀ in such an assay, and the maximal inhibitory threshold is the IC₉₀ in such an assay. In certain embodiments, FcεR-induced CD63 expression on myeloid lineage cells (basophils) is measured in a whole human blood assay. The methods for performing such an assay are known to those of skill in the art, and may be found, for example in Crawford J J, et al. J Med Chem 2018; 6:2227-2245. In some embodiments, the active inhibitory threshold is the IC₅₀, or the maximal inhibitory threshold is the IC₉₀, or both, for inhibiting myeloid lineage cells as determined by an in vitro cell assay evaluating FcεR-induced CD63 expression on myeloid lineage cells (basophils) in a whole human blood assay; wherein the assay is run with at a minimum of three separate blood donors, such as between three to ten blood donors, for example four, five, six, seven, eight, nine, or ten blood donors; and the IC₅₀, or IC₉₀, or both, as applicable, is the mean of the multiple trials. In some embodiments, the subject is orally administered a sufficient amount of a BTK inhibitor to reach a CSF concentration greater than 20% below the maximal inhibitory threshold for inhibition of myeloid lineage cell activation. For example, if the maximal inhibitory threshold were 100 ng/ml, 20% below said threshold is 80 ng/mL, and the CSF concentration would be greater than 80 ng/mL. In some embodiments, the subject is orally administered a sufficient amount of a BTK inhibitor to reach a CSF concentration between 20% to 250% of the maximal inhibitory threshold for inhibition of myeloid lineage cell activation. For example if the maximal inhibitory threshold were 100 ng/mL, between 20% to 250% of said threshold would be between 20 ng/ml and 250 ng/ml. In some embodiments, the subject is orally administered a sufficient amount of a BTK inhibitor to reach a CSF concentration between 20% to 225%, between 25% to 200%, between 30% to 190%, or between 40% to 170% of the maximal inhibitory threshold for inhibition of myeloid lineage cell activation. In some embodiments, the BTK inhibitor is fenebrutinib or a pharmaceutically acceptable salt thereof, and the subject is orally administered a sufficient amount of a BTK inhibitor to reach a CSF concentration of at least 11 ng/ml. In some embodiments, the subject is orally administered a sufficient amount of a BTK inhibitor to reach a CSF concentration of at least 15 ng/mL, or at least 20 ng/ml, or at least 30 ng/ml, or at least 33 ng/ml, or at least 43.5 ng/mL. In some embodiments, the subject is orally administered a sufficient amount of a BTK inhibitor to reach a CSF concentration between 11 ng/ml to 80 ng/mL, or between 15 ng/mL and 80 ng/mL, or between 30 ng/mL and 80 ng/mL, or between 33 ng/mL and 75 ng/mL. In some embodiments, the CSF concentration is reached at least 12 weeks after beginning oral administration of the BTK inhibitor. In some embodiments, the CSF concentration is reached at least 8 weeks after beginning oral administration of the BTK inhibitor. In some embodiments, the CSF concentration is reached at least 4 weeks after beginning oral administration of the BTK inhibitor.

In certain embodiments of the methods and uses provided herein, the CSF concentration of the BTK inhibitor is greater than the active inhibitory threshold for inhibition of BTK. In some embodiments, the CSF concentration of the BTK inhibitor is greater than the maximal inhibitory threshold for inhibition of BTK. In some embodiments, the active inhibitory threshold, or maximal inhibitory threshold, or both, for inhibiting BTK is determined by an in vitro cell assay. In some such embodiments, the in vitro assay evaluates Btk Y223 autophosphorylation in blood. In some embodiments, the active inhibitory threshold is the IC₅₀ in such an assay, and the maximal inhibitory threshold is the IC₉₀ in such an assay. In certain embodiments, Btk Y223 autophosphorylation is measured in a whole human blood assay. The methods for performing such an assay are known to those of skill in the art, and may be found, for example in Crawford J J, et al. J Med Chem 2018; 6:2227-2245. In some embodiments, the active inhibitory threshold is the IC₅₀, or the maximal inhibitory threshold is the IC₉₀, or both, for inhibiting Btk Y223 autophosphorylation in blood in a whole human blood assay; wherein the assay is run with at a minimum of three separate blood donors, such as between three to ten blood donors, for example four, five, six, seven, eight, nine, or ten blood donors; and the IC₅₀, or IC₉₀, or both, as applicable, is the mean of the multiple trials. In some embodiments, the subject is orally administered a sufficient amount of a BTK inhibitor to reach a CSF concentration greater than 20% below the maximal inhibitory threshold for inhibition of BTK. For example, if the maximal inhibitory threshold were 100 ng/mL, 20% below said threshold is 80 ng/ml, and the CSF concentration would be greater than 80 ng/mL. In some embodiments, the subject is orally administered a sufficient amount of a BTK inhibitor to reach a CSF concentration between 20% to 250% of the maximal inhibitory threshold for inhibition of BTK. For example if the maximal inhibitory threshold were 100 ng/ml, between 20% to 250% of said threshold would be between 20 ng/mL and 250 ng/mL. In some embodiments, the subject is orally administered a sufficient amount of a BTK inhibitor to reach a CSF concentration between 20% to 225%, between 25% to 200%, between 30% to 190%, or between 40% to 170% of the maximal inhibitory threshold for inhibition of BTK. In some embodiments, the BTK inhibitor is fenebrutinib or a pharmaceutically acceptable salt thereof, and the subject is orally administered a sufficient amount of a BTK inhibitor to reach a CSF concentration of at least 11 ng/mL. In some embodiments, the subject is orally administered a sufficient amount of a BTK inhibitor to reach a CSF concentration of at least 15 ng/mL, or at least 20 ng/mL, or at least 30 ng/ml, or at least 33 ng/mL, or at least 43.5 ng/mL. In some embodiments, the subject is orally administered a sufficient amount of a BTK inhibitor to reach a CSF concentration between 11 ng/ml to 80 ng/mL, or between 15 ng/mL and 80 ng/mL, or between 30 ng/ml and 80 ng/mL, or between 33 ng/ml and 75 ng/mL. In some embodiments, the CSF concentration is reached at least 12 weeks after beginning oral administration of the BTK inhibitor. In some embodiments, the CSF concentration is reached at least 8 weeks after beginning oral administration of the BTK inhibitor. In some embodiments, the CSF concentration is reached at least 4 weeks after beginning oral administration of the BTK inhibitor.

Further provided herein are methods of treating MS in a subject in need thereof, wherein the method comprises orally administering to the subject a sufficient amount of a BTK inhibitor to reach a CSF to blood plasma concentration ratio of at least 2%. Similarly, provided herein is a BTK inhibitor for use in treating MS in a subject in need thereof, wherein the treatment comprises orally administering to the subject a sufficient amount of a BTK inhibitor to reach a CSF to blood plasma concentration ratio of at least 2%. In some embodiments of the methods and uses herein, the CSF to blood plasma concentration ratio is at least 2.5%, at least 3%, at least 3.5%, at least 4%, at least 4.5%, or at least 5%, or is between 2% to 14%, between 2% to 12%, between 3% to 12%, between 3% to 10%, or between 4% to 8%. The percentage based ratio may also be expressed as relative numbers, for example 5% may be expressed as 1:20. In some embodiments, the concentration of the BTK inhibitor in the CSF and blood plasma is evaluated in CSF and blood plasma samples taken on the same day, from the same patient. In some embodiments, the ratio is reached within 4 weeks of beginning administration of the BTK inhibitor, or within 8 weeks of beginning administration of the BTK inhibitor, or within 12 weeks of beginning administration of the BTK inhibitor. In some embodiments, the MS is RMS. In some embodiments, the MS is PPMS. In some embodiments, the BTK inhibitor is fenebrutinib or a pharmaceutically acceptable salt thereof. In some embodiments, the subject is orally administered 200 mg fenebrutinib twice daily, comprising a total daily dose of 400 mg, or an equivalent amount of a pharmaceutically acceptable salt thereof. In some embodiments, the subject is orally administered fenebrutinib. In some embodiments, the subject is a human.

In some embodiments, the CSF concentration is greater than at least two of the active inhibitory threshold for inhibition of B-cell activation; the active inhibitory threshold for inhibition of myeloid lineage cell activation; and the active inhibitory threshold for inhibition of BTK. In some embodiments, all three are met. Thus, embodiments combining embodiments relating to each of B-cell activation inhibition, myeloid lineage cell activation inhibition, and BTK inhibition as described within, as combinations of any two, or all three, or any multiples, are contemplated herein. In some embodiments, embodiments directed to CSF to blood plasma concentration ratio are combined, singly or multiply, with any of the other CSF concentration embodiments including the inhibitory threshold embodiments as described herein.

### F. Statistical Significance

In some embodiments of the methods and compounds for use provided herein, the comparison discussed (such as reduction in number, or increase in odds, or rate change) is significant, and is measured in comparison to another subject (or subjects) with RMS who is not administered fenebrutinib or a pharmaceutically acceptable salt thereof (including, for example, administered a non-active placebo). In some such embodiments, for example, administration of fenebrutinib or pharmaceutically acceptable salt thereof significantly reduces one or more MRI markers of RMS disease activity in the brain compared to placebo. These MRI markers may include, independently, the number of new gadolinium enhancing T1 lesions, and the number of new or enlarging T2-weighted lesions. The MRI markers may also include T1 hypointense lesions. In some such embodiments, a comparison which is statistically significant is one in which the p-value for such comparison is <0.05. Thus, in some embodiments, administration of 200 mg fenebrutinib or an equivalent amount of a pharmaceutically acceptable salt thereof to a subject with RMS in need thereof significantly reduced the number of new gadolinium enhancing T1 lesions as measured at 12 weeks, or as measured at 8 weeks, or as measured at 4 weeks, after beginning administration. In some embodiments, administration of 200 mg fenebrutinib or an equivalent amount of a pharmaceutically acceptable salt thereof to a subject with RMS in need thereof significantly reduced the number of new or enlarging T2-weighted lesions as measured at 12 weeks, or as measured at 8 weeks, or as measured at 4 weeks, after beginning administration. In some embodiments, the administration of 200 mg fenebrutinib or an equivalent amount of a pharmaceutically acceptable salt thereof to a subject with RMS in need thereof significantly reduced the rate of developing new gadolinium enhancing T1 lesions, or significantly reduced the rate of developing new or enlarging T2-weighted lesions, or significantly increased the odds of being free from both new gadolinium enhancing T1 lesions and new or enlarging T2-weighted lesions. In some embodiments, the administration of 200 mg fenebrutinib or an equivalent amount of a pharmaceutically acceptable salt thereof to a subject with RMS in need thereof significantly reduced the number of new T1 hypointense lesions as measured at 12 weeks, or as measured at 8 weeks, or as measured at 4 weeks, after beginning administration. In some embodiments, the administration of 200 mg fenebrutinib or an equivalent amount of a pharmaceutically acceptable salt thereof to a subject with RMS in need thereof significantly reduced the rate of generating new T1 hypointense lesions as measured at 12 weeks, or as measured at 8 weeks, or as measured at 4 weeks, after beginning administration. In some embodiments, the comparison is relative to the same subject before beginning administration of fenebrutinib or a pharmaceutically acceptable salt thereof, for example as evaluated in the same subject in the immediate 12 weeks, immediate 6 months, or immediate 12 months before beginning administration of fenebrutinib or a pharmaceutically acceptable salt thereof. In some embodiments, the comparison is relative to another subject that is not administered an RMS therapy.

Each of the embodiments for said methods, uses, and treatments described herein may be combined in various permutations, where logically feasible, without limit.

### ENUMERATED EMBODIMENTS

Embodiment 1. A method of treating relapsing multiple sclerosis (RMS) in a subject in need thereof, comprising administering to the subject about 200 mg fenebrutinib twice daily, or an equivalent amount of a pharmaceutically acceptable salt thereof, and wherein the treatment comprises: reducing the total number of new gadolinium enhancing T1 lesions in the subject over 12 weeks after beginning administration, as evaluated at 4, 8, and 12 weeks.

Embodiment 2. A method of treating RMS in a subject in need thereof, comprising administering to the subject about 200 mg fenebrutinib twice daily, or an equivalent amount of a pharmaceutically acceptable salt thereof, and wherein the treatment comprises: reducing the total number of new gadolinium enhancing T1 lesions at 8 weeks after beginning administration.

Embodiment 3. A method of treating RMS in a subject in need thereof, comprising administering to the subject about 200 mg fenebrutinib twice daily, or an equivalent amount of a pharmaceutically acceptable salt thereof, and wherein the treatment comprises: reducing the total number of new gadolinium enhancing T1 lesions as evaluated at weeks 8 and 12 after beginning administration.

Embodiment 4. A method of treating RMS in a subject in need thereof, comprising administering to the subject about 200 mg fenebrutinib twice daily, or an equivalent amount of a pharmaceutically acceptable salt thereof, and wherein the treatment comprises: reducing the total number of new or enlarging T2-weighted lesions over 12 weeks after beginning administration, as evaluated at 4, 8, and 12 weeks.

Embodiment 5. A method of treating RMS in a subject in need thereof, comprising administering to the subject about 200 mg fenebrutinib twice daily, or an equivalent amount of a pharmaceutically acceptable salt thereof, and wherein the treatment comprises: reducing the total number of new or enlarging T2-weighted lesions at 8 weeks after beginning administration.

Embodiment 6. A method of treating RMS in a subject in need thereof, comprising administering to the subject about 200 mg fenebrutinib twice daily, or an equivalent amount of a pharmaceutically acceptable salt thereof, and wherein the treatment comprises: reducing the total number of new or enlarging T2-weighted lesions as evaluated at weeks 8 and 12 after beginning administration.

Embodiment 7. The method of any one of Embodiments 1 to 6, wherein the reduction is at least 60%.

Embodiment 8. A method of treating RMS in a subject in need thereof, comprising administering to the subject about 200 mg fenebrutinib twice daily, or an equivalent amount of a pharmaceutically acceptable salt thereof, and wherein the treatment comprises: reducing the rate of developing new gadolinium enhancing T1 lesions.

Embodiment 9. A method of treating RMS in a subject in need thereof, comprising administering to the subject about 200 mg fenebrutinib twice daily, or an equivalent amount of a pharmaceutically acceptable salt thereof, and wherein the treatment comprises: preventing the development of new gadolinium enhancing T1 lesions.

Embodiment 10. A method of treating RMS in a subject in need thereof, comprising administering to the subject about 200 mg fenebrutinib twice daily, or an equivalent amount of a pharmaceutically acceptable salt thereof, and wherein the treatment comprises: reducing the rate of developing new or enlarging T2-weighted lesions.

Embodiment 11. A method of treating RMS in a subject in need thereof, comprising administering to the subject about 200 mg fenebrutinib twice daily, or an equivalent amount of a pharmaceutically acceptable salt thereof, and wherein the treatment comprises: preventing the development of new or enlarging T2-weighted lesions.

Embodiment 12. A method of treating RMS in a subject in need thereof, comprising administering to the subject about 200 mg fenebrutinib twice daily, or an equivalent amount of a pharmaceutically acceptable salt thereof, and wherein the treatment comprises: increasing the odds that a subject will be free of both (a) any new gadolinium enhancing T1 lesions; and (b) any new or enlarging T2-weighted lesions.

Embodiment 13. The method of Embodiment 12, wherein the subject is at least two fold more likely to be free of both (a) any new gadolinium enhancing T1 lesions; and (b) any new or enlarging T2-weighted lesions, over the first 12 weeks after beginning administration.

Embodiment 14. The method of any one of Embodiments 1 to 13, wherein 200 mg fenebrutinib is administered orally twice daily.

Embodiment 15. The method of any one of Embodiments 1 to 14, wherein 200 mg fenebrutinib is orally administered twice daily as two tablets each comprising 200 mg fenebrutinib.

Embodiment 16. The method of any one of Embodiments 1 to 14, wherein 200 mg fenebrutinib is orally administered twice daily as four tablets each comprising 100 mg fenebrutinib.

Embodiment 17. Fenebrutinib or a pharmaceutically acceptable salt thereof for use in the treatment of relapsing multiple sclerosis (RMS) in a subject in need thereof, comprising administering to the subject about 200 mg fenebrutinib twice daily, or an equivalent amount of a pharmaceutically acceptable salt thereof, and wherein the treatment comprises: reducing the total number of new gadolinium enhancing T1 lesions in the subject over 12 weeks after beginning administration, as evaluated at 4, 8, and 12 weeks.

Embodiment 18. Fenebrutinib or a pharmaceutically acceptable salt thereof for use in the treatment RMS in a subject in need thereof, comprising administering to the subject about 200 mg fenebrutinib twice daily, or an equivalent amount of a pharmaceutically acceptable salt thereof, and wherein the treatment comprises: reducing the total number of new gadolinium enhancing T1 lesions at 8 weeks after beginning administration.

Embodiment 19. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises: reducing the total number of new gadolinium enhancing T1 lesions as evaluated at weeks 8 and 12 after beginning administration.

Embodiment 20. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises: reducing the total number of new or enlarging T2-weighted lesions over 12 weeks after beginning administration, as evaluated at 4, 8, and 12 weeks.

Embodiment 21. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises: reducing the total number of new or enlarging T2-weighted lesions at 8 weeks after beginning administration.

Embodiment 22. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises: reducing the total number of new or enlarging T2-weighted lesions as evaluated at weeks 8 and 12 after beginning administration.

Embodiment 23. Fenebrutinib or an equivalent amount of a pharmaceutically acceptable salt thereof for use in any one of Embodiments 17 to 22, wherein the reduction is at least 60%.

Embodiment 24. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises: reducing the rate of developing new gadolinium enhancing T1 lesions.

Embodiment 25. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises: preventing the development of new gadolinium enhancing T1 lesions.

Embodiment 26. Fenebrutinib or a pharmaceutically acceptable salt thereof for use in the treatment RMS in a subject in need thereof, comprising administering to the subject about 200 mg fenebrutinib twice daily, or an equivalent amount of a pharmaceutically acceptable salt thereof, and wherein the treatment comprises: reducing the rate of developing new or enlarging T2-weighted lesions.

Embodiment 27. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises: preventing the development of new or enlarging T2-weighted lesions.

Embodiment 28. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises: increasing the odds that a subject will be free of both (a) any new gadolinium enhancing T1 lesions; and (b) any new or enlarging T2-weighted lesions.

Embodiment 29. Fenebrutinib or an equivalent amount of a pharmaceutically acceptable salt thereof for use in Embodiment 28, wherein the subject is at least two fold more likely to be free of both (a) any new gadolinium enhancing T1 lesions; and (b) any new or enlarging T2-weighted lesions, over the first 12 weeks after beginning administration.

Embodiment 30. Fenebrutinib or an equivalent amount of a pharmaceutically acceptable salt thereof for use in any one of Embodiments 17 to 29, wherein 200 mg fenebrutinib is administered orally twice daily.

Embodiment 31. Fenebrutinib or an equivalent amount of a pharmaceutically acceptable salt thereof for use in any one of Embodiments 17 to 30, wherein 200 mg fenebrutinib is orally administered twice daily as two tablets each comprising 200 mg fenebrutinib.

Embodiment 32. Fenebrutinib or an equivalent amount of a pharmaceutically acceptable salt thereof for use in any one of Embodiments 17 to 30, wherein 200 mg fenebrutinib is orally administered twice daily as four tablets each comprising 100 mg fenebrutinib.

Embodiment 33. A compound for use in the manufacture of a medicament for treatment of relapsing multiple sclerosis (RMS) in a subject in need thereof, wherein the compound is fenebrutinib or a pharmaceutically acceptable salt thereof, and the treatment comprises the method of any one of Embodiments 1 to 16.

Embodiment 34. A method of treating Multiple Sclerosis (MS) in a subject in need thereof, comprising:
orally administering to the subject a sufficient amount of a BTK inhibitor to reach a cerebrospinal fluid (CSF) concentration greater than the active inhibitory threshold for inhibition of B-cell activation, inhibition of myeloid lineage cell activation, or inhibition of BTK, or any combination thereof, for said BTK inhibitor.

Embodiment 35. The method of Embodiment 34, wherein the CSF concentration is greater than the active inhibitory threshold for inhibition of B-cell activation.

Embodiment 36. The method of Embodiment 34, wherein the CSF concentration is greater than the active inhibitory threshold for inhibition of myeloid lineage cell activation.

Embodiment 37. The method of Embodiment 34, wherein the CSF concentration is greater than the active inhibitory threshold for inhibition of BTK.

Embodiment 38. The method of Embodiment 34, wherein the CSF concentration is greater than at least two of the active inhibitory threshold for inhibition of B-cell activation, the active inhibitory threshold for inhibition of myeloid lineage cell activation, or the active inhibitory threshold for inhibition of BTK.

Embodiment 39. The method of Embodiment 34, wherein the CSF concentration is greater than all three of the active inhibitory threshold for inhibition of B-cell activation, the active inhibitory threshold for inhibition of myeloid lineage cell activation, and the active inhibitory threshold for inhibition of BTK.

Embodiment 40. The method of any one of Embodiments 34 to 39, wherein the active inhibitory threshold for inhibition of B-cell activation is the IC₅₀ of B-cell activation inhibition as evaluated using an in vitro cell assay measuring a B-cell activation marker; the active inhibitory threshold for inhibition of myeloid lineage cell activation is the IC₅₀ of a myeloid lineage cell activation inhibition as evaluated using an in vitro cell assay measuring a myeloid lineage cell activation marker; and the active inhibitory threshold for inhibition of BTK is the IC₅₀ of BTK inhibition as evaluated using an in vitro cell assay measuring BTK activity.

Embodiment 41. The method of any one of Embodiments 34 to 40, wherein the subject is orally administered a sufficient amount of a BTK inhibitor to reach a CSF concentration greater than 20% below (a) the maximal inhibitory threshold for inhibition of B-cell activation, (b) the maximal inhibitory threshold for inhibition of myeloid lineage cell activation, or (c) the maximal inhibitory threshold for inhibition of BTK, or any combination thereof, for said BTK inhibitor.

Embodiment 42. The method of any one of Embodiments 34 to 41, wherein the subject is orally administered a sufficient amount of a BTK inhibitor to reach a CSF concentration greater than (a) the maximal inhibitory threshold for inhibition of B-cell activation, (b) the maximal inhibitory threshold for inhibition of myeloid lineage cell activation, or (c) the maximal inhibitory threshold for inhibition BTK, or any combinations thereof, for said BTK inhibitor.

Embodiment 43. The method of Embodiment 41 or 42, wherein the CSF concentration is greater than 20% below the maximal inhibitory threshold for inhibition of B-cell activation.

Embodiment 44. The method of any one of Embodiments 41 to 43, wherein the CSF concentration is greater than the maximal inhibitory threshold for inhibition of B-cell activation.

Embodiment 45. The method of Embodiment 41 or 42, wherein the CSF concentration is greater than 20% below the maximal inhibitory threshold for inhibition of myeloid lineage cell activation.

Embodiment 46. The method of any one of Embodiments 41, 42, or 45, wherein the CSF concentration is greater than the maximal inhibitory threshold for inhibition of myeloid lineage cell activation.

Embodiment 47. The method of Embodiment 41 or 42, wherein the CSF concentration is greater than 20% below the maximal inhibitory threshold for inhibition of BTK.

Embodiment 48. The method of any one of Embodiments 41, 42, or 47, wherein the CSF concentration is greater than the maximal inhibitory threshold for inhibition of BTK.

Embodiment 49. The method of any one of Embodiments 41 to 56, wherein the CSF concentration is greater than the maximal inhibitory threshold for all three of inhibition of B-cell activation, inhibition of myeloid lineage cell activation, or inhibition of BTK.

Embodiment 50. The method of any one of Embodiments 41 to 49, wherein the maximal inhibitory threshold for inhibition of B-cell activation is the IC₉₀ of B-cell activation inhibition as evaluated using an in vitro cell assay measuring a B-cell activation marker; the maximal inhibitory threshold for inhibition of myeloid lineage cell activation is the IC₉₀ of a myeloid lineage cell activation inhibition as evaluated using an in vitro cell assay measuring a myeloid lineage cell activation marker; and the maximal inhibitory threshold for inhibition of BTK is the IC₉₀ of BTK inhibition as evaluated using an in vitro cell assay measuring BTK autophosphorylation activity.

Embodiment 51. The method of any one of Embodiments 34 to 50, wherein the BTK inhibitor is fenebrutinib or a pharmaceutically acceptable salt thereof.

Embodiment 52. The method of Embodiment 51, wherein CSF concentration is at least 11 ng/mL.

Embodiment 53. The method of Embodiment 51, wherein CSF concentration is at least 33 ng/mL.

Embodiment 54. The method Embodiment 53, wherein CSF concentration is at least 43.5 ng/mL.

Embodiment 55. The method of any one of Embodiments 34 to 54, wherein the CSF concentration is reached at least 12 weeks after beginning oral administration of the BTK inhibitor.

Embodiment 56. The method of any one Embodiments 34 to 55, wherein the MS is Primary Progressive MS (PPMS).

Embodiment 57. The method of any one Embodiments 34 to 56, wherein the MS is Relapsing MS (RMS).

Embodiment 58. The method of any one of Embodiments 34 to 57, wherein the CSF-to-plasma ratio of the BTK inhibitor in the subject is at least 4%.

Embodiment 59. The method of any one of Embodiments 34 to 58, wherein the subject is orally administered 200 mg of the BTK inhibitor fenebrutinib twice daily, for a total daily dose of 400 mg fenebrutinib, or an equivalent amount of a pharmaceutically acceptable salt of fenebrutinib.

Embodiment 60. A method of treating RMS in a subject in need thereof, comprising administering to the subject about 200 mg fenebrutinib twice daily, or an equivalent amount of a pharmaceutically acceptable salt thereof, and wherein the treatment comprises: reducing the total number of new T1 hypointense lesions in the subject.

Embodiment 61. The method of Embodiment 60, wherein the total number of new T1 hypointense lesions is reduced at 12 weeks after beginning administration.

Embodiment 62. The method of Embodiment 60, wherein the total number of new T1 hypointense lesions is reduced at 8 weeks after beginning administration.

Embodiment 63. The method of Embodiment 60, wherein the total number of new T1 hypointense lesions is reduced at 4 weeks after beginning administration

Embodiment 64. The method of Embodiment 60, wherein the total number of new T1 hypointense lesions is reduced at 12 weeks after beginning administration, as evaluated at 4, 8, and 12 weeks.

Embodiment 65. The method of any one of Embodiments 60 to 64, wherein the reduction is at least 40%.

Embodiment 66. The method of any one of Embodiments 60 to 62, or 64, wherein the reduction is at least 50%.

Embodiment 67. The method of any one of Embodiments 60, 62, or 64, wherein the reduction is at least 60%.

Embodiment 68. A method of treating RMS in a subject in need thereof, comprising administering to the subject about 200 mg fenebrutinib twice daily, or an equivalent amount of a pharmaceutically acceptable salt thereof, and wherein the treatment comprises: reducing the rate of developing new T1 hypointense lesions.

Embodiment 69. The method of Embodiment 68, wherein the rate is evaluated over a period of 4 weeks, 8 weeks, or 12 weeks, after beginning administration.

Embodiment 70. A method of treating RMS in a subject in need thereof, comprising administering to the subject about 200 mg fenebrutinib twice daily, or an equivalent amount of a pharmaceutically acceptable salt thereof, and wherein the treatment comprises: preventing the development of new T1 hypointense lesions.

Embodiment 71. The method of Embodiment 70, wherein the development is prevented over a period of 4 weeks, 8 weeks, or 12 weeks, after beginning administration.

Embodiment 72. The method of any one of Embodiments 34 to 71, wherein 200 mg of fenebrutinib is administered orally twice daily.

Embodiment 73. The method of any one of Embodiments 34 to 71, wherein 200 mg fenebrutinib is orally administered twice daily as two tablets each comprising 200 mg fenebrutinib.

Embodiment 74. The method of any one of Embodiments 34 to 71, wherein 200 mg fenebrutinib is orally administered twice daily as four tablets each comprising 100 mg fenebrutinib.

Embodiment 75. The method of any one of Embodiments 2, 3, 5, or 6, wherein the reduction is at least 80%.

Embodiment 76. The method of any one of Embodiments 2, 3, 5, or 6, wherein the reduction is at least 90%.

Embodiment 77. Fenebrutinib or an equivalent amount of a pharmaceutically acceptable salt thereof for use in any one of Embodiments 18, 19, 21, or 22, wherein the reduction is at least 80%.

Embodiment 78. Fenebrutinib or an equivalent amount of a pharmaceutically acceptable salt thereof for use in any one of Embodiments 18, 19, 21, or 22, wherein the reduction is at least 90%.

Embodiment 79. The method of Embodiment 12, or fenebrutinib or an equivalent amount of a pharmaceutically acceptable salt thereof for use of Embodiment 28, wherein the odds are increased at least three fold.

Embodiment 80. Fenebrutinib or an equivalent amount of a pharmaceutically acceptable salt thereof for use in any of the methods of Embodiments 34 to 74.

Embodiment 81. The method of any one of Embodiments 1 to 16, 34 to 74, or 79, wherein the comparison is to the same subject evaluated prior to beginning administration of fenebrutinib or a pharmaceutically acceptable salt thereof.

Embodiment 82. The method of Embodiment 81, wherein the same subject is evaluated in the 12 weeks, 6 months, or 12 months immediately prior to beginning administration of fenebrutinib or a pharmaceutically acceptable salt thereof.

Embodiment 83. The method of any one of Embodiments 1 to 16, 34 to 74, or 79, wherein the comparison is to a different subject who is not administered fenebrutinib or a pharmaceutically acceptable salt thereof.

Embodiment 84. The method of any one of Embodiments 1 to 16, 34 to 74, or 79, wherein the comparison is to the same subject evaluated prior to beginning administration of fenebrutinib or a pharmaceutically acceptable salt thereof.

Embodiment 85. Fenebrutinib or an equivalent amount of a pharmaceutically acceptable salt thereof for use in any one of Embodiments 17 to 33, or 75 to 79, wherein the comparison is to the same subject evaluated prior to beginning administration of fenebrutinib or a pharmaceutically acceptable salt thereof.

Embodiment 86. Fenebrutinib or an equivalent amount of a pharmaceutically acceptable salt thereof for use in Embodiment 89, wherein the same subject is evaluated in the 12 weeks, 6 months, or 12 months immediately prior to beginning administration of fenebrutinib or a pharmaceutically acceptable salt thereof.

Embodiment 87. Fenebrutinib or an equivalent amount of a pharmaceutically acceptable salt thereof for use in any one of Embodiments 17 to 33, or 75 to 79, wherein the comparison is to the same subject evaluated prior to beginning administration of fenebrutinib or a pharmaceutically acceptable salt thereof.

Embodiment 88. A compound for use in the manufacture of a medicament for treatment in a subject in need thereof, wherein the compound is fenebrutinib or a pharmaceutically acceptable salt thereof, and the treatment comprises the method of any one of Embodiments 34 to 74, 79, or 81 to 84.

### EXAMPLES

The present disclosure will be more fully understood by reference to the following examples. They should not, however, be construed as limiting the scope of the invention.

### Example 1: A Randomized, Double-Blind, Placebo-Controlled Study to Investigate the Efficacy of Fenebrutinib in Relapsing Multiple Sclerosis (Phase II)

This Phase II study was undertaken to evaluate the effect of fenebrutinib on brain magnetic resonance imaging (MRI) in participants with RMS. Participants were randomized to receive either fenebrutinib or placebo. In addition to MRI evaluations, participants were also monitored for other clinical outcomes of disease, including disability progression and MS relapse, as described more fully below.

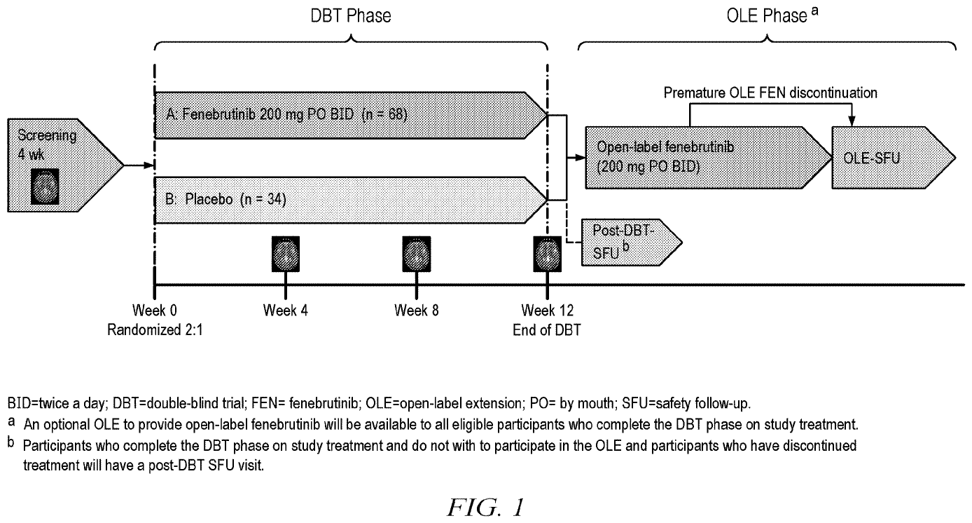

Study Design: This clinical trial comprised a double-blind treatment (DBT) phase, where participants were randomized in a 2:1 ratio to receive either 200 mg BID oral fenebrutinib or non-active placebo, administered for 12 weeks. Participants were directed to self-administer two 100 mg tablets in the morning and two 100 mg tablets in the evening by mouth, for a total dose of 400 mg of fenebrutinib (or placebo) every day. A diagram of the study design is provided in FIG. 1.

A total of 109 adults aged 18-55 with RMS were enrolled in the study: 73 in the fenebrutinib arm, and 36 in the placebo arm. Three patients in the fenebrutinib arm did not complete the treatment phase; all patients in the placebo arm completed the treatment phase. Accordingly, the analysis of outcome for the double blind phase of the study included 70 patients in the fenebrutinib arm, and 36 patients in the placebo arm.

MRI Measurements: The primary efficacy endpoint for this study was the total number of new gadolinium-enhancing T1 (T1Gd+) lesions on brain MRI measured at Weeks 4, 8, and 12 of administration. The secondary efficacy outcomes were (1) the total number of new or enlarging T2-weighted lesions on brain MRI measured at Weeks 4, 8, and 12, and (2) the proportion of participants free from any new Gd-enhancing T1 lesions and new or enlarging T2-weighted lesions observed on brain MRI at Weeks 4, 8, and 12. Radiologic evaluation for the primary efficacy parameter was performed using a standardized MRI protocol at screening, and at Weeks 4, 8, and 12 for each participant.

Exploratory Endpoints: Exploratory endpoints included the total number of new T1-hypointense lesions on MRI at weeks 4, 8, and 12; the fenebrutinib concentration in CSF collected at baseline and at or after Week 12; and plasma pharmacokinetics.

Clinical Outcome Assessments: Disability was measured using the Expanded Disability Status Scale (EDSS). The EDSS is based on a standardized neurological examination, incorporating functional systems (visual, brainstem, pyramidal, cerebellar, sensory, bowel and bladder, and cerebral [or mental]) that are rated and then scored as a functional systems score (FSS), and ambulation, which is scored as ambulation score. Each FSS is an ordinal clinical rating scale ranging from 0 to 5 or 6 and an ambulation score that is rated from 0 to 16. These ratings are then used in conjunction with observations, as well as information, concerning ambulation and use of assistive devices to determine the total EDSS score. The EDSS is a disability scale that ranges in 0.5-point steps from 0 (normal) to 10.0 (death) (Kurtzke 1983; Kappos 2011). All FSS, ambulation score, and total EDSS scores were captured electronically.

MS Relapse Assessment: For this study, a protocol-defined relapse was defined as the occurrence of new or worsening neurological symptoms attributed to MS and immediately preceded by a relatively stable or improving neurological state of at least 30 days. Symptoms must have persisted for >24 hours and not be attributable to confounding clinical factors (e.g., fever, infection, injury, adverse reactions to concomitant medications). The new or worsening neurological symptoms must have been accompanied by objective neurological worsening consistent with an increase of at least one of the following:
Half a step (0.5 point) on the EDSS
Two points on one of the selected FSS listed below.
One point on two or more of the selected FSS listed below

The change must have affected the following selected FSS: pyramidal, ambulation, cerebellar, brainstem, sensory, or visual. Episodic spasms, sexual dysfunction, fatigue, mood change, or bladder or bowel urgency or incontinence did not suffice to establish a relapse.

Patients were eligible for this study if they met certain eligibility criteria, which included:
Age 18 to 55 years inclusive at the time of signing Informed Consent Form
A diagnosis of RMS in accordance with the revised 2017 McDonald Criteria (Thompson et al. 2018) and one of the following:
   At least two documented clinical relapses within the last 2 years or one documented clinical relapse within 12 months of screening (but not within the 30 days prior to screening)
   Documented evidence of the presence of at least one T1 Gd+ lesion on MRI in the 6 months prior to randomization (may include the screening MRI)
      Note: RMS may include active secondary progressive MS as defined by Lublin 2014.
Expanded Disability Status Scale (EDSS) at screening from 0 to 5.5 points

Participants were excluded from the study if they met certain exclusion criteria, which included:
Disease duration of >10 years from the onset of symptoms and an EDSS score at screening <2.0
A diagnosis of primary progressive MS or non-active secondary progressive MS.
Presence of other neurological disorders that could interfere with the diagnosis of MS or with the assessments of efficacy or safety during the study
Previous treatment with fenebrutinib or another BTK inhibitor for any indication
Treatment with strong CYP3A4 inhibitors, strong or moderate CYP3A4 inducers, within 7 days or 5 drug-elimination half-lives (whichever is longer) prior to randomization
Treatment with CYP3A4 substrates with a narrow therapeutic window within 7 days or 5 drug-elimination half-lives (whichever is longer) prior to randomization

Statistical Considerations: The study was 90% powered to detect a 60% reduction in the primary endpoint, assuming 0.7 new T1 Gd+ lesions per scan in the placebo arm and 3% dropout rate.

Baseline Disease Characteristics: A summary of baseline disease characteristics is provided in Table 1.

TABLE 1
Baseline Disease Characteristics

| | Fenebrutinib | | Placebo | | All Participants | |
|---|---|---|---|---|---|---|
| | (N = 73) | | (N = 36) | | (N = 109) | |
| Age, mean (SD), years | 38.6 | (8.5) | 39.8 | (7.7) | 39.0 | (8.2) |
| Female, n (%) | 52 | (71.2%) | 26 | (72.2%) | 78 | (71.6%) |
| Disease Modifying Therapies Status | | | | | | |
| n | 73 | | 36 | | 109 | |
| Used Before Trial | 18 | (24.7%) | 17 | (47.2%) | 35 | (32.1%) |
| No Use Before Trial | 55 | (75.3%) | 19 | (52.8%) | 74 | (67.9%) |
| Baseline T1Gd + Status | | | | | | |
| n | 73 | | 36 | | 109 | |
| Present, n (%) | 14 | (19.2%) | 7 | (19.4%) | 21 | (19.3%) |
| Absent, n (%) | 59 | (80.8%) | 29 | (80.6%) | 88 | (80.7%) |
| Number of T1Gd + Lesions | | | | | | |
| n | 73 | | 36 | | 109 | |
| Mean (SD) | 0.74 | (2.26) | 0.72 | (2.31) | 0.73 | (2.27) |
| Median | 0.00 | | 0.00 | | 0.00 | |
| Min-Max | 0.0-14.0 | | 0.0-13.0 | | 0.0-14.0 | |
| Patients with T1 Gd + Lesions, n (%) | 14 | (19.2%) | 7 | (19.4%) | 21 | (19.3%) |
| Number of T2-weighted Lesions | | | | | | |
| n | 73 | | 36 | | 109 | |
| Mean (SD) | 45.03 | (28.46) | 52.22 | (33.09) | 47.40 | (30.11) |
| Median | 42.00 | | 46.00 | | 43.00 | |
| Min-Max | 6.0-140.0 | | 7.0-153.0 | | 6.0-153.0 | |
| Baseline EDSS | | | | | | |
| n | 73 | | 36 | | 109 | |
| Mean (SD) | 2.53 | (1.15) | 2.71 | (1.35) | 2.59 | (1.22) |
| Median | 2.50 | | 3.00 | | 2.50 | |
| Min-Max | 0.0-5.5 | | 0.0-5.5 | | 0.0-5.5 | |
| Number of T1 hypointense lesions | | | | | | |
| Mean (SD) | 21.1 | (21.7) | 26.2 | (28.4) | 22.8 | (24.1) |
| Time since diagnosis | | | | | | |
| Mean (SD), years | 4.9 | (5.9) | 5.9 | (7.1) | 5.2 | (6.3) |

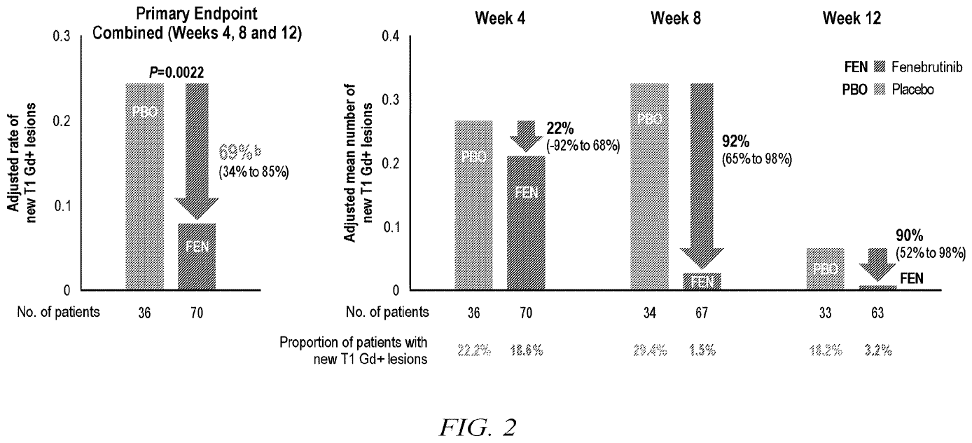

Results: Primary Endpoint (Total Number of New T1 Gd+ Lesions at Weeks 4, 8, and 12, combined): The primary endpoint of this study was met. In the treatment arm (n=70), a 69% relative reduction in new T1 Gd+ lesions was observed, compared to placebo, with a p value of 0.0022. A summary of the primary endpoint analysis is provided in Table 2. A supplementary analysis of the primary endpoint, breaking down the results by visit, is provided in Tables 3 and 4. FIG. 2 provides bar graphs illustrating some of the data in these tables, and providing additional analyses. In this figure, the large arrows indicate the relative reduction (95% confidence interval) of lesions. The proportion of patients with new T1 Gd+ lesions is the number of participants that have a new lesion at that specific visit alone divided by the number of participants with a scan at that specific visit alone at the indicated week. Few patients had new lesions after week four of treatment with fenebrutinib. There was a 90% reduction and only 3.2% of patients with new T1 Gd+ Lesions lesions at Week 12. Overall, 38% of FEN pts (n=73) and 33% of PBO pts (n=36) had an adverse event (AE). No serious AEs or deaths were reported.

TABLE 2
Summary of Total Number of New T1 Gd + Lesions
at Weeks 4, 8, and 12 combined (Primary Endpoint)

| | Fenebrutinib | Placebo |
|---|---|---|
| | (N = 70) | (N = 36) |
| Total Number of New T1Gd + Lesions | 29 | 38 |
| Total number of scans | 201 | 103 |
| New T1Gd + Lesion rate, unadjusted^{a} | 0.144 | 0.369 |
| New T1Gd + Lesion rate, adjusted^{a} | 0.077 | 0.245 |
| Rate Ratio, adjusted | 0.313 | |
| 95% CI | (0.149, 0.658) | |
| Relative Reduction | 69% | |
| 95% CI | (34%, 85%) | |
| p-value | 0.0022 | |

^{a}The unadjusted lesion rate is the total number of lesions for all patients in the considered group divided by the total number of scans. The adjusted rate and rate ratio are estimated from a Negative Binomial regression model for the total number of events adjusted for the following covariates: presence of any T1Gd + lesions at baseline. The log of number of MRI scans is included as an offset.

TABLE 3
Total Number of New T1 Gd + Lesions at each of Week 4, Week
8, and Week 12 Visits (Supplementary analysis of primary endpoint)

| Visit | Fenebrutinib | Placebo |
|---|---|---|
| Baseline | | |
| n | 73 | 36 |
| Mean (SD) | 0.74 (2.26) | 0.72 (2.31) |
| Min-max | 0-14 | 0-13 |
| Week 4 | | |
| n | 70 | 36 |
| Mean (SD) | 0.343 (0.849) | 0.389 (0.871) |
| Min-max | 0-4 | 0-3 |
| Week 8 | | |
| n | 67 | 34 |
| Mean (SD) | 0.045 (0.367) | 0.441 (0.960) |
| Min-max | 0-3 | 0-5 |
| Week 12 | | |
| n | 63 | 33 |
| Mean (SD) | 0.032 (0.177) | 0.273 (0.674) |
| Min-max | 0-1 | 0-3 |

TABLE 4
Relative Reduction of total number of new
T1 Gd + lesions, broken down by visit

| | Relative Reduction | 95% Confidence Interval |
|---|---|---|
| Week 4 | 22% | −92%, 68% |
| Week 8 | 92% | 65%, 98% |
| Week 12 | 90% | 52%, 98% |
| Weeks 8 and 12 | 92% | |

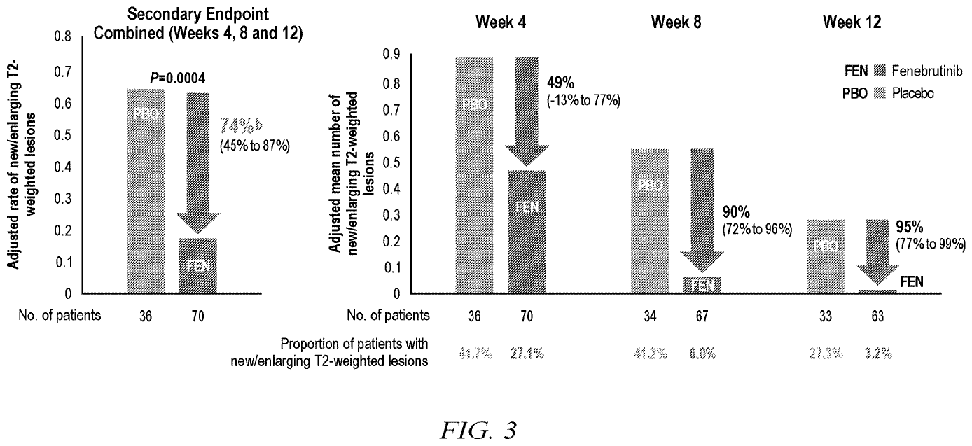

Results: Secondary Endpoint-Total Number of New or Enlarging T2-Weighted Lesions at Weeks 4, 8, and 12 combined: This secondary endpoint was met, and a summary of this secondary endpoint analysis is provided in Table 5. A supplementary analysis of the secondary endpoint, breaking down the results by visit, is provided in Tables 6 and 7. FIG. 3 provides bar graphs illustrating some of the data in these tables and providing additional analyses. In this figure, the large arrows indicate the relative reduction (95% confidence interval) of lesions. The proportion of patients with new/enlarging T2-weighted lesions is the number of participants that have a new lesion at that specific visit alone divided by the number of participants with a scan at that specific visit alone at the indicated week. There was a 95% reduction and only 3.2% of patients with new/enlarging lesions at Week 12.

TABLE 5
Summary of New or Enlarging T2-Weighted Lesions at
Weeks 4, 8, and 12 combined (Secondary Endpoint)

| | Fenebrutinib | Placebo |
|---|---|---|
| | (N = 70) | (N = 36) |
| Total Number of New or Enlarging | 82 | 90 |
| T2-Weighted Lesions | | |
| Total number of scans | 201 | 103 |
| New or Enlarging T2-Weighted | 0.408 | 0.874 |
| Lesion rate, unadjusted | | |
| New or Enlarging T2-Weighted | 0.168 | 0.634 |
| Lesion rate, adjusted | | |
| Rate Ratio, adjusted | 0.265 | |
| 95% CI | (0.128, 0.550) | |
| Relative Reduction | 73.5% | |
| 95% CI | (45.0%, 87.2%) | |
| p-value | 0.0004 | |

TABLE 6
Total Number of New or Enlarging T2-Weighted Lesions
at each of Week 4, Week 8, and Week 12 Visits
(Supplementary analysis of secondary endpoint)

| Visit | Fenebrutinib | Placebo |
|---|---|---|
| Baseline | | |
| n | 73 | 36 |
| Mean (SD) | 45.03 (28.46) | 52.22 (33.09) |
| Median | 42 | 46 |
| Min-max | 6-140 | 7-153 |
| Week 4 | | |
| n | 70 | 36 |
| Mean (SD) | 1.043 (2.990) | 1.222 (2.218) |
| Min-max | 0-21 | 0-10 |
| Week 8 | | |
| n | 67 | 34 |
| Mean (SD) | 0.104 (0.526) | 0.824 (1.359) |
| Min-max | 0-4 | 0-5 |
| Week 12 | | |
| n | 63 | 33 |
| Mean (SD) | 0.032 (0.177) | 0.545 (1.121) |
| Min-max | 0-1 | 0-5 |

TABLE 7
Relative Reduction of New or Enlarging T2-
Weighted Lesions, broken down by visit

| | Relative Reduction | 95% Confidence Interval |
|---|---|---|
| Week 4 | 49% | −13%, 77% |
| Week 8 | 90% | 72%, 96% |
| Week 12 | 95% | 77%, 99% |
| Weeks 8 and 12 | 93% | |

Results: Secondary Endpoint-Proportion of Participants Free from any new Gd+ Enhancing T1 Lesions and New or Enlarging T2-weighted Lesions at Weeks 4, 8, and 12: This secondary endpoint was also met, and a summary of the analysis provided in Table 8. A supplementary analysis of this secondary endpoint, at each of visits week 4, 8, and 12 separately, is provided in Table 9. Patients in the fenebrutinib group were four times more likely to be free from new T1 Gd+ lesions and new or enlarging T2-weighted lesions at weeks 4, 8, and 12 compared to patients in the placebo group.

TABLE 8
Proportion of patients free from any new T1 Gd +
lesions and new or enlarging T2-weighted lesions
at weeks 4, 8, and 12 (Secondary Endpoint)

| | Fenebrutinib | Placebo |
|---|---|---|
| | (N = 70) | (N = 36) |
| No. of patients who are free from | 51 (72.9%) | 18 (50.0%) |
| new T1 Gd + and New or Enlarging | | |
| T2-weighted Lesions During DBT (%) | | |
| Odds Ratio, adjusted | 4.005 | |
| 95% CI (exact) | (1.317, 13.078) | |
| p-value (exact) | 0.0117 | |

TABLE 9
Supplementary Analysis of the Secondary Endpoint: Proportion
of Participants Free From Any New T1 Gd + Lesions
and New or Enlarging T2-Weighted Lesions by Visit
(supplementary analysis of secondary endpoint)

| Visit | Fenebrutinib | Placebo |
|---|---|---|
| Overall | | |
| n (No. of patients free from T1 Gd + and | 51 | 18 |
| T2-weighted lesions) | | |
| N (No. of patients with evaluable MRI scan) | 70 | 36 |
| Proportion | 72.9% | 50.0% |
| Week 4 | | |
| n | 51 | 21 |
| N | 70 | 36 |
| Proportion | 72.9% | 58.3% |
| Week 8 | | |
| n | 63 | 20 |
| N | 67 | 34 |
| Proportion | 94.0% | 58.8% |
| Week 12 | | |
| n | 61 | 24 |
| N | 63 | 33 |
| Proportion | 96.8% | 72.7% |

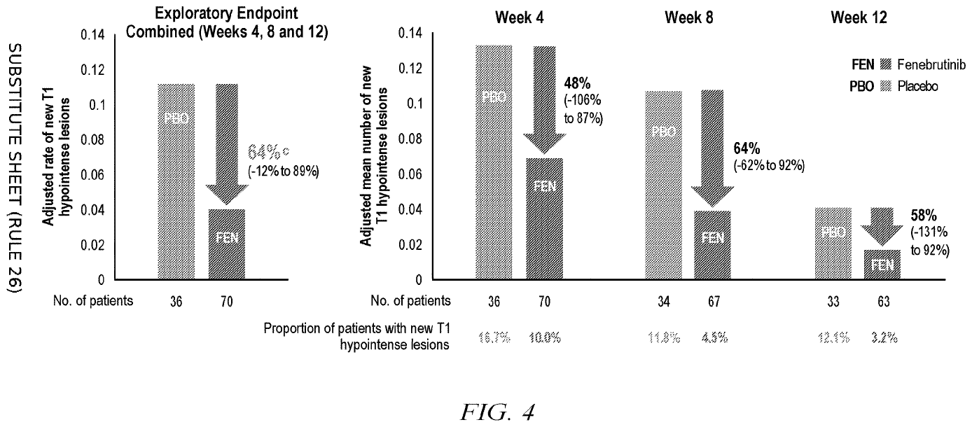

Results: Exploratory Endpoint-Hypointense T1 Lesions: The number of T1 hypointense lesions was evaluated at Week 4, 8, and 12 as an exploratory endpoint. Treatment with fenebrutinib reduced T1 hypointense lesions at all three time points, and few patients had new lesions after Week 4 of treatment, compared to placebo. The results of the analysis are provided in Table 10. FIG. 4 provides bar graphs illustrating some of the data in this table and providing additional analyses. In this figure, the large arrows indicate the relative reduction (95% confidence interval) of lesions. The proportion of patients with new T1 hypointense lesions is the number of participants that have a new lesion at that specific visit alone divided by the number of participants with a scan at that specific visit alone at the indicated week. There was a 58% reduction and only 3.2% of patients with new hypointense T1 lesions at Week 12.

TABLE 10
Relative Reduction of T1 hypointense
lesions, broken down by visit

| | Relative Reduction | 95% Confidence Interval |
|---|---|---|
| Week 4 | 48% | −106%, 87% |
| Week 8 | 64% | −62%, 92% |
| Week 12 | 58% | −131%, 92% |
| Weeks 4, 8, and 12 | 64% | −12%, 89% |

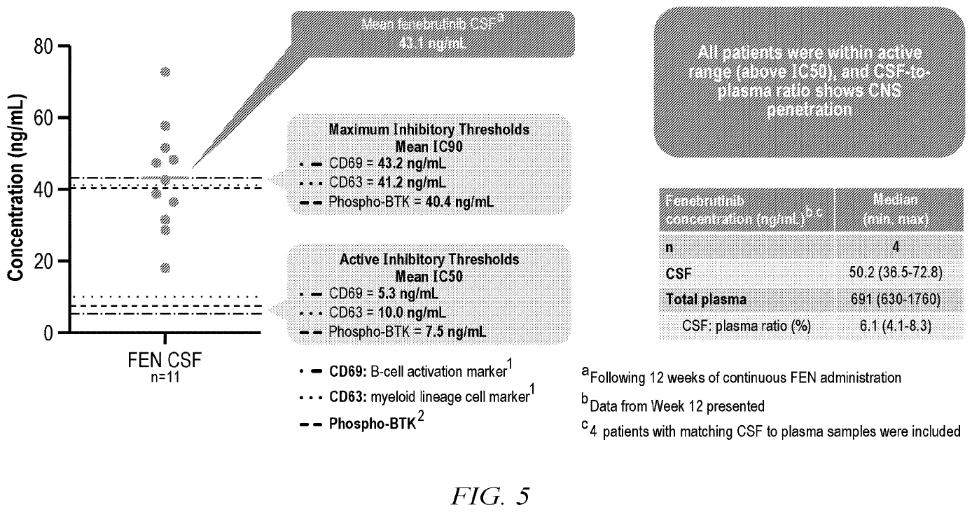

Results: Exploratory Endpoint-Evaluation of CSF: Cerebrospinal samples (CSF) were taken from 11 patients following 12 weeks of continuous fenebrutinib administration. A blood plasma sample was taken at the same time for 4 of the 11 patients. The concentration of fenebrutinib in CSF at 12 Weeks was evaluated, and presented in FIG. 5 in comparison to the IC50 of fenebrutinib (active inhibitory threshold) and the IC90 of fenebrutinib (maximal inhibitory threshold) as evaluated in different in vitro assays. From the figure, (a) the mean fenebrutinib CSF concentration is following 12 weeks of continuous fenebrutinib administration; (1) the data for CD63 and CD69 assays was previously presented as Weber M S, et al. at AAN 2021 Virtual Annual Meeting, April 17-22. (P15.091); and (2) the data for the phospho-BTK assay was previously presented in Keaney J, et al. J Neuroimmune Pharmacol 2019; 14:448-461. Analysis of fenebrutinib concentration in CSF, plasma, and the ratio for the four patients for which a plasma sample was taken is provided in Table 11.

TABLE 11
CSF and total plasma concentration in 4 patients after
12 weeks of continuous fenebrutinib administration.

| n = 4 | Median ng/mL (min, max) |
|---|---|
| CSF | 50.2 (36.5-72.8) |
| Total plasma | 691 (630-1760) |
| CSF:plasma ratio (%) | 6.1 (4.1-8.3) |

As shown by FIG. 5, the CSF fenebrutinib concentration in all 11 patients was within the active range (above the IC50 of fenebrutinib evaluated in CD63, CD69, and phospho-BTK inhibitory assays), with the mean fenebrutinib CSF concentration (43.1 ng/ml) comparable to the IC90 of fenebrutinib as evaluated in different in vitro human whole blood assays (CD69: 43.2 ng/ml; CD63: 41.2 ng/ml; phospho-BTK: 40.4 ng/mL). CD63 is a myeloid lineage cell activation marker; CD69 is a B-cell activation marker; the phospho-BTK assay evaluates target engagement through inhibition of anti-IgM induced BTK Y223 autophosphorylation in human whole blood. Procedures for performing these three assays may be found in Crawford J J, et al. J Med Chem 2018; 6:2227-2245. Fenebrutinib has a dual mechanism of action, inhibiting both B-cell and myeloid lineage cell activation. Accordingly, the data in FIG. 5 illustrates the concentration of fenebrutinib observed in the CSF after 12 weeks of continuous administration is within the active range, and comparable to the maximal inhibitory thresholds, of both mechanisms of action as evaluated by the CD63 and CD69 in vitro cell assays, and BTK target engagement as evaluated by a phoshpo-BTK in vitro assay. The concentration of fenebrutinib observed in the CSF after 12 weeks of continuous administration is also Fenebrutinib was present in the CSF at levels sufficient to reduce activation of B cells and microglia progenitor cells in vitro. The CSF-to-plasma ratio demonstrates CNS access of fenebrutinib in patients.

Results—Safety Summary: A summary of adverse events (AEs) for patients treated with fenebrutinib, and with placebo, is provided in Table 12.

TABLE 12
Summary adverse events (AEs) and serious adverse events
(SAEs) for patients enrolled in the clinical trial.

| | Fenebrutinib | Placebo |
|---|---|---|
| | (n = 73) | (n = 36) |
| Patients with ≥1 AE, n (%) | 28 (38.4) | 12 (33.3) |
| Total no. of AEs | 40 | 19 |
| Patients with ≥1, n (%) | | |
| AE with fatal outcome | 0 | 0 |
| SAE | 0 | 0 |
| AE leading to dose interruption | 0 | 0 |
| Related AE | 10 (13.7) | 2 (5.6) |
| Grade 3 AE | 2 (2.7) | 0 |
| Patients withdrawn from study | 7 (9.6) | 0 |
| due to an AE, n (%) | | |
| Most common AEs, n (%) | | |
| Abnormal hepatic enzyme levels | 4 (5.5) | 0 |
| Urinary tract infection | 4 (5.5) | 2 (5.6) |
| Headache | 3 (4.1) | 1 (2.8) |
| Nasopharyngitis | 2 (2.7) | 0 |
| Nausea | 2 (2.7) | 1 (2.8) |
| Pharyngitis | 2 (2.7) | 1 (2.8) |
| Upper abdominal pain | 2 (2.7) | 0 |

No SAEs were reported. All AEs were Grade 1 or 2, except for two Grade 3 asymptomatic liver transaminase level elevations. The observed hepatic transaminase level elevations in fenebrutinib-treated patients were reversible and asymptomatic. The rate of infections was balanced between the fenebrutinib and placebo arms. There were four AEs leading to withdrawal from the study: 4 abnormal hepatic transaminase levels (discontinuation required by protocol); 1 upper abdominal pain; nausea and headache; 1 upper abdominal pain; and 1 hypersensitivity.

What is claimed is:

1. A method of treating relapsing multiple sclerosis (RMS) in a subject in need thereof, comprising administering to the subject about 200 mg fenebrutinib twice daily, or an equivalent amount of a pharmaceutically acceptable salt thereof, and wherein the treatment comprises:
reducing the total number of new gadolinium enhancing T1 lesions in the subject over 12 weeks after beginning administration, as evaluated at 4, 8, and 12 weeks.

2. A method of treating RMS in a subject in need thereof, comprising administering to the subject about 200 mg fenebrutinib twice daily, or an equivalent amount of a pharmaceutically acceptable salt thereof, and wherein the treatment comprises:
reducing the total number of new gadolinium enhancing T1 lesions at 8 weeks after beginning administration.

3. A method of treating RMS in a subject in need thereof, comprising administering to the subject about 200 mg fenebrutinib twice daily, or an equivalent amount of a pharmaceutically acceptable salt thereof, and wherein the treatment comprises:
reducing the total number of new gadolinium enhancing T1 lesions as evaluated at weeks 8 and 12 after beginning administration.

4. A method of treating RMS in a subject in need thereof, comprising administering to the subject about 200 mg fenebrutinib twice daily, or an equivalent amount of a pharmaceutically acceptable salt thereof, and wherein the treatment comprises:
reducing the total number of new or enlarging T2-weighted lesions over 12 weeks after beginning administration, as evaluated at 4, 8, and 12 weeks.

5. A method of treating RMS in a subject in need thereof, comprising administering to the subject about 200 mg fenebrutinib twice daily, or an equivalent amount of a pharmaceutically acceptable salt thereof, and wherein the treatment comprises:
reducing the total number of new or enlarging T2-weighted lesions at 8 weeks after beginning administration.

6. A method of treating RMS in a subject in need thereof, comprising administering to the subject about 200 mg fenebrutinib twice daily, or an equivalent amount of a pharmaceutically acceptable salt thereof, and wherein the treatment comprises:
reducing the total number of new or enlarging T2-weighted lesions as evaluated at weeks 8 and 12 after beginning administration.

7. The method of any one of claims 1 to 6, wherein the reduction is at least 60%.

8. A method of treating RMS in a subject in need thereof, comprising administering to the subject about 200 mg fenebrutinib twice daily, or an equivalent amount of a pharmaceutically acceptable salt thereof, and wherein the treatment comprises:
reducing the rate of developing new gadolinium enhancing T1 lesions.

9. A method of treating RMS in a subject in need thereof, comprising administering to the subject about 200 mg fenebrutinib twice daily, or an equivalent amount of a pharmaceutically acceptable salt thereof, and wherein the treatment comprises:
preventing the development of new gadolinium enhancing T1 lesions.

10. A method of treating RMS in a subject in need thereof, comprising administering to the subject about 200 mg fenebrutinib twice daily, or an equivalent amount of a pharmaceutically acceptable salt thereof, and wherein the treatment comprises:
reducing the rate of developing new or enlarging T2-weighted lesions.

11. A method of treating RMS in a subject in need thereof, comprising administering to the subject about 200 mg fenebrutinib twice daily, or an equivalent amount of a pharmaceutically acceptable salt thereof, and wherein the treatment comprises:
preventing the development of new or enlarging T2-weighted lesions.

12. A method of treating RMS in a subject in need thereof, comprising administering to the subject about 200 mg fenebrutinib twice daily, or an equivalent amount of a pharmaceutically acceptable salt thereof, and wherein the treatment comprises:
increasing the odds that a subject will be free of both (a) any new gadolinium enhancing T1 lesions; and (b) any new or enlarging T2-weighted lesions.

13. The method of claim 12, wherein the subject is at least two fold more likely to be free of both (a) any new gadolinium enhancing T1 lesions; and (b) any new or enlarging T2-weighted lesions, over the first 12 weeks after beginning administration.

14. The method of any one of claims 1 to 13, wherein 200 mg fenebrutinib is administered orally twice daily.

15. The method of any one of claims 1 to 14, wherein 200 mg fenebrutinib is orally administered twice daily as two tablets each comprising 200 mg fenebrutinib.

16. The method of any one of claims 1 to 14, wherein 200 mg fenebrutinib is orally administered twice daily as four tablets each comprising 100 mg fenebrutinib.

17. Fenebrutinib or a pharmaceutically acceptable salt thereof for use in the treatment of relapsing multiple sclerosis (RMS) in a subject in need thereof, comprising administering to the subject about 200 mg fenebrutinib twice daily, or an equivalent amount of a pharmaceutically acceptable salt thereof, and wherein the treatment comprises:
reducing the total number of new gadolinium enhancing T1 lesions in the subject over 12 weeks after beginning administration, as evaluated at 4, 8, and 12 weeks.

18. Fenebrutinib or a pharmaceutically acceptable salt thereof for use in the treatment RMS in a subject in need thereof, comprising administering to the subject about 200 mg fenebrutinib twice daily, or an equivalent amount of a pharmaceutically acceptable salt thereof, and wherein the treatment comprises:
reducing the total number of new gadolinium enhancing T1 lesions at 8 weeks after beginning administration.

19. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises:
reducing the total number of new gadolinium enhancing T1 lesions as evaluated at weeks 8 and 12 after beginning administration.

20. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises:
reducing the total number of new or enlarging T2-weighted lesions over 12 weeks after beginning administration, as evaluated at 4, 8, and 12 weeks.

21. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises:
reducing the total number of new or enlarging T2-weighted lesions at 8 weeks after beginning administration.

22. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises:
reducing the total number of new or enlarging T2-weighted lesions as evaluated at weeks 8 and 12 after beginning administration.

23. Fenebrutinib or an equivalent amount of a pharmaceutically acceptable salt thereof for use in any one of claims 17 to 22, wherein the reduction is at least 60%.

24. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises:
reducing the rate of developing new gadolinium enhancing T1 lesions.

25. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises:
preventing the development of new gadolinium enhancing T1 lesions.

26. Fenebrutinib or a pharmaceutically acceptable salt thereof for use in the treatment RMS in a subject in need thereof, comprising administering to the subject about 200 mg fenebrutinib twice daily, or an equivalent amount of a pharmaceutically acceptable salt thereof, and wherein the treatment comprises:
reducing the rate of developing new or enlarging T2-weighted lesions.

27. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises:
preventing the development of new or enlarging T2-weighted lesions.

28. Fenebrutinib or a pharmaceutically acceptable salt thereof for use in the treatment of RMS in a subject in need thereof, comprising administering to the subject about 200 mg fenebrutinib twice daily, or an equivalent amount of a pharmaceutically acceptable salt thereof, and wherein the treatment comprises:
increasing the odds that a subject will be free of both (a) any new gadolinium enhancing T1 lesions; and (b) any new or enlarging T2-weighted lesions.

29. Fenebrutinib or an equivalent amount of a pharmaceutically acceptable salt thereof for use in claim 28, wherein the subject is at least two fold more likely to be free of both (a) any new gadolinium enhancing T1 lesions; and (b) any new or enlarging T2-weighted lesions, over the first 12 weeks after beginning administration.

30. Fenebrutinib or an equivalent amount of a pharmaceutically acceptable salt thereof for use in any one of claims 17 to 29, wherein 200 mg fenebrutinib is administered orally twice daily.

31. Fenebrutinib or an equivalent amount of a pharmaceutically acceptable salt thereof for use in any one of claims 17 to 30, wherein 200 mg fenebrutinib is orally administered twice daily as two tablets each comprising 200 mg fenebrutinib.

32. Fenebrutinib or an equivalent amount of a pharmaceutically acceptable salt thereof for use in any one of claims 17 to 30, wherein 200 mg fenebrutinib is orally administered twice daily as four tablets each comprising 100 mg fenebrutinib.

33. A compound for use in the manufacture of a medicament for treatment of relapsing multiple sclerosis (RMS) in a subject in need thereof, wherein the compound is fenebrutinib or a pharmaceutically acceptable salt thereof, and the treatment comprises the method of any one of claims 1 to 16.

34. A method of treating Multiple Sclerosis (MS) in a subject in need thereof, comprising:
orally administering to the subject a sufficient amount of a BTK inhibitor to reach a cerebrospinal fluid (CSF) concentration greater than the active inhibitory threshold for inhibition of B-cell activation, inhibition of myeloid lineage cell activation, or inhibition of BTK, or any combination thereof, for said BTK inhibitor.

35. The method of claim 34, wherein the active inhibitory threshold for inhibition of B-cell activation is the IC₅₀ of B-cell activation inhibition as evaluated using an in vitro cell assay measuring a B-cell activation marker; the active inhibitory threshold for inhibition of myeloid lineage cell activation is the IC₅₀ of a myeloid lineage cell activation inhibition as evaluated using an in vitro cell assay measuring a myeloid lineage cell activation marker; and the active inhibitory threshold for inhibition of BTK is the IC₅₀ of BTK inhibition as evaluated using an in vitro cell assay measuring BTK activity.

36. The method of any one of claims 34 to 35, wherein the subject is orally administered a sufficient amount of a BTK inhibitor to reach a CSF concentration greater than 20% below (a) the maximal inhibitory threshold for inhibition of B-cell activation, (b) the maximal inhibitory threshold for inhibition of myeloid lineage cell activation, or (c) the maximal inhibitory threshold for inhibition of BTK, or any combination thereof, for said BTK inhibitor.

37. The method of any one of claims 34 to 36, wherein the subject is orally administered 200 mg of the BTK inhibitor fenebrutinib twice daily, for a total daily dose of 400 mg fenebrutinib, or an equivalent amount of a pharmaceutically acceptable salt of fenebrutinib.

38. A method of treating RMS in a subject in need thereof, comprising administering to the subject about 200 mg fenebrutinib twice daily, or an equivalent amount of a pharmaceutically acceptable salt thereof, and wherein the treatment comprises: reducing the total number of new T1 hypointense lesions in the subject.

39. The method of claim 38, wherein the reduction is at least 40%.

40. A method of treating RMS in a subject in need thereof, comprising administering to the subject about 200 mg fenebrutinib twice daily, or an equivalent amount of a pharmaceutically acceptable salt thereof, and wherein the treatment comprises: reducing the rate of developing new T1 hypointense lesions.

41. A method of treating RMS in a subject in need thereof, comprising administering to the subject about 200 mg fenebrutinib twice daily, or an equivalent amount of a pharmaceutically acceptable salt thereof, and wherein the treatment comprises: preventing the development of new T1 hypointense lesions.
